(19) United States
(12) Patent Application Publication
(10) Pub. No.: US 2021/0139467 A1
(43) Pub. Date: May 13, 2021

(54) MODULATORS OF INDOLEAMINE 2,3-DIOXYGENASE

(71) Applicant: GLAXOSMITHKLINE INTELLECTUAL PROPERTY DEVELOPMENT LIMITED, Brenford, Middlesex (GB)

(72) Inventors: Martha Alicia DE LA ROSA, Research Triangle Park, NC (US); Wieslaw Mieczyslaw KAZMIERSKI, Research Triangle Park, NC (US)

(21) Appl. No.: 16/618,461

(22) PCT Filed: Jun. 27, 2018
(86) PCT No.: PCT/IB2018/054762

§ 371 Date: Dec. 2, 2019

Related U.S. Application Data
(60) Provisional application No. 62/666,772, filed on May 4, 2018.
(60) Provisional application No. 62/525,794, filed on Jun. 28, 2017.

(51) Int. Cl. C07D 405/14; C07D 405/12; C07D 213/74; C07D 285/08; C07D 417/12

(52) U.S. Cl. CPC C07D 405/14; C07D 405/12; C07D 417/12; C07D 285/08; C07D 213/74

(57) ABSTRACT

Provided are IDO inhibitor compounds of Formula I and pharmaceutically acceptable salts thereof, their pharmaceutical compositions, their methods of preparation, and methods for their use in the prevention and/or treatment of diseases.

[chemical structure]

### FIELD OF THE INVENTION

Compounds, methods and pharmaceutical compositions for the prevention and/or treatment of HIV; including the prevention of the progression of AIDS and general immunosuppression, by administering certain indoleamine 2,3-dioxygenase compounds in therapeutically effective amounts are disclosed. Methods for preparing such compounds and methods of using the compounds and pharmaceutical compositions thereof are also disclosed.

### BACKGROUND OF THE INVENTION

Indoleamine-2,3-dioxygenase 1 (IDO1) is a heme-containing enzyme that catalyzes the oxidation of the indole ring of tryptophan to produce N-formyl kynurenine, which is rapidly and constitutively converted to kynurenine (Kyn) and a series of downstream metabolites. IDO1 is the rate limiting step of this kynurenine pathway of tryptophan metabolism and expression of IDO1 is inducible in the context of inflammation. Stimuli that induce IDO1 include viral or bacterial products, or inflammatory cytokines associated with infection, tumors, or sterile tissue damage. Kyn and several downstream metabolites are immunosuppressive: Kyn is antiproliferative and proapoptotic to T cells and NK cells (Munn, Shafizadeh et al. 1999, Frumento, Rotondo et al. 2002) while metabolites such as 3-hydroxy anthranilic acid (3-HAA) or the 3-HAA oxidative dimerization product cinnabarinic acid (CA) inhibit phagocyte function (Sekkai, Guittet et al. 1997), and induce the differentiation of immunosuppressive regulatory T cells (Treg) while inhibiting the differentiation of gut-protective IL-17 or IL-22-producing CD4+ T cells (Th17 and Th22)(Favre, Mold et al. 2010). IDO1 induction, among other mechanisms, is likely important in limiting immunopathology during active immune responses, in promoting the resolution of immune responses, and in promoting fetal tolerance. However in chronic settings, such as cancer, or chronic viral or bacterial infection, IDO1 activity prevents clearance of tumor or pathogen and if activity is systemic, IDO1 activity may result in systemic immune dysfunction (Boasso and Shearer 2008, Li, Huang et al. 2012). In addition to these immunomodulatory effects, metabolites of IDO1 such as Kyn and quinolinic acid are also known to be neurotoxic and are observed to be elevated in several conditions of neurological dysfunction and depression. As such, IDO1 is a therapeutic target for inhibition in a broad array of indications, such as to promote tumor clearance, enable clearance of intractable viral or bacterial infections, decrease systemic immune dysfunction manifest as persistent inflammation during HIV infection or immunosuppression during sepsis, and prevent or reverse neurological conditions.

### IDO1 and Persistent Inflammation in HIV Infection:

Despite the success of antiretroviral therapy (ART) in suppressing HIV replication and decreasing the incidence of AIDS-related conditions, HIV-infected patients on ART have a higher incidence of non-AIDS morbidities and mortality than their uninfected peers. These non-AIDS conditions include cancer, cardiovascular disease, osteoporosis, liver disease, kidney disease, frailty, and neurocognitive dysfunction (Deeks 2011). Several studies indicate that non-AIDS morbidity/mortality is associated with persistent inflammation, which remains elevated in HIV-infected patients on ART as compared to peers (Deeks 2011). As such, it is hypothesized that persistent inflammation and immune dysfunction despite virologic suppression with ART is a cause of these non-AIDS-defining events (NADEs).

HIV infects and kills CD4+ T cells, with particular preference for cells like those CD4+ T cells that reside in the lymphoid tissues of the mucosal surfaces (Mattapallil, Douek et al. 2005). The loss of these cells combined with the inflammatory response to infection result in a perturbed relationship between the host and all pathogens, including HIV itself, but extending to pre-existing or acquired viral infections, fungal infections, and resident bacteria in the skin and mucosal surfaces. This dysfunctional host:pathogen relationship results in the over-reaction of the host to what would typically be minor problems as well as permitting the outgrowth of pathogens among the microbiota. The dysfunctional host:pathogen interaction therefore results in increased inflammation, which in turn leads to deeper dysfunction, driving a vicious cycle. As inflammation is thought to drive non-AIDS morbidity/mortality, the mechanisms governing the altered host:pathogen interaction are therapeutic targets.

IDO1 expression and activity are increased during untreated and treated HIV infection as well as in primate models of SIV infection (Boasso, Vaccari et al. 2007, Favre, Lederer et al. 2009, Byakwaga, Boum et al. 2014, Hunt, Sinclair et al. 2014, Tenorio, Zheng et al. 2014). IDO1 activity, as indicated by the ratio of plasma levels of enzyme substrate and product (Kyn/Tryp or K:T ratio), is associated with other markers of inflammation and is one of the strongest predictors of non-AIDS morbidity/mortality (Byakwaga, Boum et al. 2014, Hunt, Sinclair et al. 2014, Tenorio, Zheng et al. 2014). In addition, features consistent with the expected impact of increased IDO1 activity on the immune system are major features of HIV and SIV induced immune dysfunction, such as decreased T cell proliferative response to antigen and imbalance of Treg:Th17 in systemic and intestinal compartments (Favre, Lederer et al. 2009, Favre, Mold et al. 2010). As such, we and others hypothesize that IDO1 plays a role in driving the vicious cycle of immune dysfunction and inflammation associated with non-AIDS morbidity/mortality. Thus, we propose that inhibiting IDO1 will reduce inflammation and decrease the risk of NADEs in ART-suppressed HIV-infected persons.

### IDO1 and Persistent Inflammation Beyond HIV

As described above, inflammation associated with treated chronic HIV infection is a likely driver of multiple end organ diseases [Deeks 2011]. However, these end organ diseases are not unique to HIV infection and are in fact the common diseases of aging that occur at earlier ages in the HIV-infected population. In the uninfected general population inflammation of unknown etiology is a major correlate of morbidity and mortality [Pinti, 2016 #88]. Indeed many of the markers of inflammation are shared, such as IL-6 and CRP. If, as hypothesized above, IDO1 contributes to persistent inflammation in the HIV-infected population by inducing immune dysfunction in the GI tract or systemic tissues, then IDO1 may also contribute to inflammation and therefore end organ diseases in the broader population. These inflammation associated end organ diseases are exemplified by cardiovascular diseases, metabolic syndrome, liver disease (NAFLD, NASH), kidney disease, osteoporosis, and neurocognitive impairment. Indeed, the IDO1 pathway has links in the literature to liver disease (Vivoli abstracts at Italian Assoc. for the Study of the Liver Conference 2015], diabetes [Baban, 2010 #89], chronic kidney disease [Schefold, 2009 #90], cardiovascular disease [Mangge, 2014 #92; Mangge, 2014 #91], as well as general aging and all cause mortality [Pertovaara, 2006 #93]. As such, inhibition of IDO1 may have application in decreasing inflammation in the general population to decrease the incidence of specific end organ diseases associated with inflammation and aging.

### IDO1 and Oncology

IDO expression can be detected in a number of human cancers (for example; melanoma, pancreatic, ovarian, AML, CRC, prostate and endometrial) and correlates with poor prognosis (Munn 2011). Multiple immunosuppressive roles have been ascribed to the action of IDO, including the induction of Treg differentiation and hyper-activation, suppression of Teff immune response, and decreased DC function, all of which impair immune recognition and promote tumor growth (Munn 2011). IDO expression in human brain tumors is correlated with reduced survival. Orthotropic and transgenic glioma mouse models demonstrate a correlation between reduced IDO expression and reduced Treg infiltration and a increased long term survival (Wainwright, Balyasnikova et al. 2012). In human melanoma a high proportion of tumors (33 of 36 cases) displayed elevated IDO suggesting an important role in establishing an immunosuppressive tumor microenvironment (TME) characterized by the expansion, activation and recruitment of MDSCs in a Treg-dependent manner (Holmgaard, Zamarin et al. 2015). Additionally, host IDO expressing immune cells have been identified in the draining lymph nodes and in the tumors themselves (Mellor and Munn 2004). Hence, both tumor and host-derived IDO are believed to contribute to the immune suppressed state of the TME.

The inhibition of IDO was one of the first small molecule drug strategies proposed for re-establishment of an immunogenic response to cancer (Mellor and Munn 2004). The d-enantiomer of 1-methyl tryptophan (D-1MTor indoximod) was the first IDO inhibitor to enter clinical trials. While this compound clearly does inhibit the activity of IDO, it is a very weak inhibitor of the isolated enzyme and the in vivo mechanism(s) of action for this compound are still being elucidated. Investigators at Incyte optimized a hit compound obtained from a screening process into a potent and selective inhibitor with sufficient oral exposure to demonstrate a delay in tumor growth in a mouse melanoma model (Yue, Douty et al. 2009). Further development of this series led to INCB204360 which is a highly selective for inhibition of IDO-1 over IDO-2 and TDO in cell lines transiently transfected with either human or mouse enzymes (Liu, Shin et al. 2010). Similar potency was seen for cell lines and primary human tumors which endogenously express IDO1 (IC50s˜3-20 nM). When tested in co-culture of DCs and naïve CD4⁺CD25⁻ T cells, INCB204360 blocked the conversion of these T cells into CD4⁺FoxP3⁺ Tregs. Finally, when tested in a syngeneic model (PAN02 pancreatic cells) in immunocompetent mice, orally dosed INCB204360 provided a significant dose-dependent inhibition of tumor growth, but was without effect against the same tumor implanted in immune-deficient mice. Additional studies by the same investigators have shown a correlation of the inhibition of IDO1 with the suppression of systemic kynurenine levels and inhibition of tumor growth in an additional syngeneic tumor model in immunocompetent mice. Based upon these preclinical studies, INCB24360 entered clinical trials for the treatment of metastatic melanoma (Beatty, O'Dwyer et al. 2013).

In light of the importance of the catabolism of tryptophan in the maintenance of immune suppression, it is not surprising that overexpression of a second tryptophan metabolizing enzyme, TDO2, by multiple solid tumors (for example, bladder and liver carcinomas, melanomas) has also been detected. A survey of 104 human cell lines revealed 20/104 with TDO expression, 17/104 with IDO1 and 16/104 expressing both (Pilotte, Larrieu et al. 2012). Similar to the inhibition of IDO1, the selective inhibition of TDO2 is effective in reversing immune resistance in tumors overexpressing TDO2 (Pilotte, Larrieu et al. 2012). These results support TDO2 inhibition and/or dual TDO2/IDO1 inhibition as a viable therapeutic strategy to improve immune function.

Multiple pre-clinical studies have demonstrated significant, even synergistic, value in combining IDO-1 inhibitors in combination with T cell checkpoint modulating mAbs to CTLA-4, PD-1, and GITR. In each case, both efficacy and related PD aspects of improved immune activity/function were observed in these studies across a variety of murine models (Balachandran, Cavnar et al. 2011, Holmgaard, Zamarin et al. 2013, M. Mautino 2014, Wainwright, Chang et al. 2014). The Incyte IDO1 inhibitor (INCB204360, epacadostat) has been clinically tested in combination with a CTLA4 blocker (ipilimumab), but it is unclear that an effective dose was achieved due to dose-limited adverse events seen with the combination. In contrast recently released data for an on-going trial combining epacadostat with Merck's PD-1 mAb (pembrolizumab) demonstrated improved tolerability of the combination allowing for higher doses of the IDO1 inhibitor. There have been several clinical responses across various tumor types which is encouraging. However, it is not yet known if this combination is an improvement over the single agent activity of pembrolizumab (Gangadhar, Hamid et al. 2015). Similarly, Roche/Genentech are advancing NGL919/GDC-0919 in combination with both mAbs for PD-L1 (MPDL3280A, Atezo) and OX-40 following the recent completion of a phase 1a safety and PK/PD study in patients with advanced tumors.

### IDO1 and Chronic Infections

IDO1 activity generates kynurenine pathway metabolites such as Kyn and 3-HAA that impair at least T cell, NK cell, and macrophage activity (Munn, Shafizadeh et al. 1999, Frumento, Rotondo et al. 2002) (Sekkai, Guittet et al. 1997, Favre, Mold et al. 2010). Kyn levels or the Kyn/Tryp ratio are elevated in the setting of chronic HIV infection (Byakwaga, Boum et al. 2014, Hunt, Sinclair et al. 2014, Tenorio, Zheng et al. 2014), HBV infection (Chen, Li et al. 2009), HCV infection (Larrea, Riezu-Boj et al. 2007, Asghar, Ashiq et al. 2015), and TB infection (Suzuki, Suda et al. 2012) and are associated with antigen-specific T cell dysfunction (Boasso, Herbeuval et al. 2007, Boasso, Hardy et al. 2008, Loughman and Hunstad 2012, Ito, Ando et al. 2014, Lepiller, Soulier et al. 2015). As such, it is thought that in these cases of chronic infection, IDO1-mediated inhibition of the pathogen-specific T cell response plays a role in the persistence of infection, and that inhibition of IDO1 may have a benefit in promoting clearance and resolution of infection.

### IDO1 and Sepsis

IDO1 expression and activity are observed to be elevated during sepsis and the degree of Kyn or Kyn/Tryp elevation corresponded to increased disease severity, including mortality (Tattevin, Monnier et al. 2010, Darcy, Davis et al. 2011). In animal models, blockade of IDO1 or IDO1 genetic knockouts protected mice from lethal doses of LPS or from mortality in the cecal ligation/puncture model (Jung, Lee et al. 2009, Hoshi, Osawa et al. 2014). Sepsis is characterized by an immunosuppressive phase in severe cases (Hotchkiss, Monneret et al. 2013), potentially indicating a role for IDO1 as a mediator of immune dysfunction, and indicating that pharmacologic inhibition of IDO1 may provide a clinical benefit in sepsis.

### IDO1 and Neurological Disorders

In addition to immunologic settings, IDO1 activity is also linked to disease in neurological settings (reviewed in Lovelace Neuropharmacology 2016 (Lovelace, Varney et al. 2016)). Kynurenine pathway metabolites such as 3-hydroxykynurenine and quinolinic acid are neurotoxic, but are balanced by alternative metabolites kynurenic acid or picolinic acid, which are neuroprotective. Neurodegenerative and psychiatric disorders in which kynurenine pathway metabolites have been demonstrated to be associated with disease include multiple sclerosis, motor neuron disorders such as amyotrophic lateral sclerosis, Huntington's disease, Parkinson's disease, Alzheimer's disease, major depressive disorder, schizophrenia, anorexia (Lovelace, Varney et al. 2016). Animal models of neurological disease have shown some impact of weak IDO1 inhibitors such as 1-methyltryptophan on disease, indicating that IDO1 inhibition may provide clinical benefit in prevention or treatment of neurological and psychiatric disorders.

It would therefore be an advance in the art to discover IDO inhibitors that effective the balance of the aforementioned properties as a disease modifying therapy in chronic HIV infections to decrease the incidence of non-AIDS morbidity/mortality; and/or a disease modifying therapy to prevent mortality in sepsis; and/or an immunotherapy to enhance the immune response to HIV, HBV, HCV and other chronic viral infections, chronic bacterial infections, chronic fungal infections, and to tumors; and/or for the treatment of depression or other neurological/neuropsychiatric disorders.

Asghar, K., M. T. Ashiq, B. Zulfiqar, A. Mahroo, K. Nasir and S. Murad (2015). “Indoleamine 2,3-dioxygenase expression and activity in patients with hepatitis C virus-induced liver cirrhosis.” Exp Ther Med 9(3): 901-904.
Balachandran, V. P., M. J. Cavnar, S. Zeng, Z. M. Bamboat, L. M. Ocuin, H. Obaid, E. C. Sorenson, R. Popow, C. Ariyan, F. Rossi, P. Besmer, T. Guo, C. R. Antonescu, T. Taguchi, J. Yuan, J. D. Wolchok, J. P. Allison and R. P. Dematteo (2011). “Imatinib potentiates antitumor T cell responses in gastrointestinal stromal tumor through the inhibition of Ido.” Nature Medicine 17(9): 1094-1100.
Beatty, G. L., P. J. O'Dwyer, J. Clark, J. G. Shi, R. C. Newton, R. Schaub, J. Maleski, L. Leopold and T. Gajewski (2013). “Phase I study of the safety, pharmacokinetics (PK), and pharmacodynamics (PD) of the oral inhibitor of indoleamine 2,3-dioxygenase (IDO1) INCB024360 in patients (pts) with advanced malignancies.” ASCO Meeting Abstracts 31(15_suppl): 3025.
Boasso, A., A. W. Hardy, S. A. Anderson, M. J. Dolan and G. M. Shearer (2008). “HIV-induced type I interferon and tryptophan catabolism drive T cell dysfunction despite phenotypic activation.” PLoS One 3(8): e2961.
Boasso, A., J. P. Herbeuval, A. W. Hardy, S. A. Anderson, M. J. Dolan, D. Fuchs and G. M. Shearer (2007). “HIV inhibits CD4+ T-cell proliferation by inducing indoleamine 2,3-dioxygenase in plasmacytoid dendritic cells.” Blood 109(8): 3351-3359.
Boasso, A. and G. M. Shearer (2008). “Chronic innate immune activation as a cause of HIV-1 immunopathogenesis.” Clin Immunol 126(3): 235-242.
Boasso, A., M. Vaccari, A. Hryniewicz, D. Fuchs, J. Nacsa, V. Cecchinato, J. Andersson, G. Franchini, G. M. Shearer and C. Chougnet (2007). “Regulatory T-cell markers, indoleamine 2,3-dioxygenase, and virus levels in spleen and gut during progressive simian immunodeficiency virus infection.” J Virol 81(21): 11593-11603.
Byakwaga, H., Y. Boum, 2nd, Y. Huang, C. Muzoora, A. Kembabazi, S. D. Weiser, J. Bennett, H. Cao, J. E. Haberer, S. G. Deeks, D. R. Bangsberg, J. M. McCune, J. N. Martin and P. W. Hunt (2014). “The kynurenine pathway of tryptophan catabolism, CD4+ T-cell recovery, and mortality among HIV-infected Ugandans initiating antiretroviral therapy.” J Infect Dis 210(3): 383-391.
Chen, Y. B., S. D. Li, Y. P. He, X. J. Shi, Y. Chen and J. P. Gong (2009). “Immunosuppressive effect of IDO on T cells in patients with chronic hepatitis B*.” Hepatol Res 39(5): 463-468.
Darcy, C. J., J. S. Davis, T. Woodberry, Y. R. McNeil, D. P. Stephens, T. W. Yeo and N. M. Anstey (2011). “An observational cohort study of the kynurenine to tryptophan ratio in sepsis: association with impaired immune and microvascular function.” PLoS One 6(6): e21185.
Deeks, S. G. (2011). “HIV infection, inflammation, immunosenescence, and aging.” Annu Rev Med 62: 141-155.
Favre, D., S. Lederer, B. Kanwar, Z. M. Ma, S. Proll, Z. Kasakow, J. Mold, L. Swainson, J. D. Barbour, C. R. Baskin, R. Palermo, I. Pandrea, C. J. Miller, M. G. Katze and J. M. McCune (2009). “Critical loss of the balance between Th17 and T regulatory cell populations in pathogenic SIV infection.” PLoS Pathop 5(2): e1000295.
Favre, D., J. Mold, P. W. Hunt, B. Kanwar, P. Loke, L. Seu, J. D. Barbour, M. M. Lowe, A. Jayawardene, F. Aweeka, Y. Huang, D. C. Douek, J. M. Brenchley, J. N. Martin, F. M. Hecht, S. G. Deeks and J. M. McCune (2010). “Tryptophan catabolism by indoleamine 2,3-dioxygenase 1 alters the balance of TH17 to regulatory T cells in HIV disease.” Sci Transl Med 2(32): 32ra36.
Frumento, G., R. Rotondo, M. Tonetti, G. Damonte, U. Benatti and G. B. Ferrara (2002). “Tryptophan-derived catabolites are responsible for inhibition of T and natural killer cell proliferation induced by indoleamine 2,3-dioxygenase.” J Exp Med 196(4): 459-468.
Gangadhar, T., O. Hamid, D. Smith, T. Bauer, J. Wasser, J. Luke, A. Balmanoukian, D. Kaufman, Y. Zhao, J. Maleski, L. Leopold and T. Gajewski (2015). “Preliminary results from a Phase I/II study of epacadostat (incb024360) in combination with pembrolizumab in patients with selected advanced cancers.” Journal for Immuno Therapy of Cancer 3(Suppl 2): O7.

### Holmgaard, R. B., D. Zamarin, Y. Li, B. Gasmi, D. H. Munn, J. P. Allison, T. Merghoub and J. D. Wolchok (2015). “Tumor-Expressed IDO Recruits and Activates MDSCs in a Treg-Dependent Manner.” Cell Reports 13(2): 412-424.

Holmgaard, R. B., D. Zamarin, D. H. Munn, J. D. Wolchok and J. P. Allison (2013). “Indoleamine 2,3-dioxygenase is a critical resistance mechanism in antitumor T cell immunotherapy targeting CTLA-4.” Journal of Experimental Medicine 210(7): 1389-1402. Hoshi, M., Y. Osawa, H. Ito, H. Ohtaki, T. Ando, M. Takamatsu, A. Hara, K. Saito and M. Seishima (2014). “Blockade of indoleamine 2,3-dioxygenase reduces mortality from peritonitis and sepsis in mice by regulating functions of CD11b+ peritoneal cells.” Infect Immun 82(11): 4487-4495.
Hotchkiss, R. S., G. Monneret and D. Payen (2013). “Sepsis-induced immunosuppression: from cellular dysfunctions to immunotherapy.” Nat Rev Immunol 13(12): 862-874.
Hunt, P. W., E. Sinclair, B. Rodriguez, C. Shive, B. Clagett, N. Funderburg, J. Robinson, Y. Huang, L. Epling, J. N. Martin, S. G. Deeks, C. L. Meinert, M. L. Van Natta, D. A. Jabs and M. M. Lederman (2014). “Gut epithelial barrier dysfunction and innate immune activation predict mortality in treated HIV infection.” J Infect Dis 210(8): 1228-1238.
Ito, H., T. Ando, K. Ando, T. Ishikawa, K. Saito, H. Moriwaki and M. Seishima (2014). “Induction of hepatitis B virus surface antigen-specific cytotoxic T lymphocytes can be up-regulated by the inhibition of indoleamine 2, 3-dioxygenase activity.” Immunology 142(4): 614-623.
Jung, I. D., M. G. Lee, J. H. Chang, J. S. Lee, Y. I. Jeong, C. M. Lee, W. S. Park, J. Han, S. K. Seo, S. Y. Lee and Y. M. Park (2009). “Blockade of indoleamine 2,3-dioxygenase protects mice against lipopolysaccharide-induced endotoxin shock.” J Immunol 182(5): 3146-3154.
Larrea, E., J. I. Riezu-Boj, L. Gil-Guerrero, N. Casares, R. Aldabe, P. Sarobe, M. P. Civeira, J. L. Heeney, C. Rollier, B. Verstrepen, T. Wakita, F. Borras-Cuesta, J. J. Lasarte and J. Prieto (2007). “Upregulation of indoleamine 2,3-dioxygenase in hepatitis C virus infection.” J Virol 81(7): 3662-3666.
Lepiller, Q., E. Soulier, Q. Li, M. Lambotin, J. Barths, D. Fuchs, F. Stoll-Keller, T. J. Liang and H. Barth (2015). “Antiviral and Immunoregulatory Effects of Indoleamine-2,3-Dioxygenase in Hepatitis C Virus Infection.” J Innate Immun 7(5): 530-544.
Li, L., L. Huang, H. P. Lemos, M. Mautino and A. L. Mellor (2012). “Altered tryptophan metabolism as a paradigm for good and bad aspects of immune privilege in chronic inflammatory diseases.” Front Immunol 3: 109.
Liu, X., N. Shin, H. K. Koblish, G. Yang, Q. Wang, K. Wang, L. Leffet, M. J. Hansbury, B. Thomas, M. Rupar, P. Waeltz, K. J. Bowman, P. Polam, R. B. Sparks, E. W. Yue, Y. Li, R. Wynn, J. S. Fridman, T. C. Burn, A. P. Combs, R. C. Newton and P. A. Scherle (2010). “Selective inhibition of IDO1 effectively regulates mediators of antitumor immunity.” Blood 115(17): 3520-3530.
Loughman, J. A. and D. A. Hunstad (2012). “Induction of indoleamine 2,3-dioxygenase by uropathogenic bacteria attenuates innate responses to epithelial infection.” J Infect Dis 205(12): 1830-1839.
Lovelace, M. D., B. Varney, G. Sundaram, M. J. Lennon, C. K. Lim, K. Jacobs, G. J. Guillemin and B. J. Brew (2016). “Recent evidence for an expanded role of the kynurenine pathway of tryptophan metabolism in neurological diseases.” Neuropharmacology.
M. Mautino, C. J. L., N. Vahanian, J. Adams, C. Van Allen, M. D. Sharma, T. S. Johnson and D.H. Munn (2014). “Synergistic antitumor effects of combinatorial immune checkpoint inhibition with anti-PD-1/PD-L antibodies and the IDO pathway inhibitors NLG919 and indoximod in the context of active immunotherapy.” April 2014 AACR Meeting Poster #5023.
Mattapallil, J. J., D. C. Douek, B. Hill, Y. Nishimura, M. Martin and M. Roederer (2005). “Massive infection and loss of memory CD4+ T cells in multiple tissues during acute SIV infection.” Nature 434(7037): 1093-1097.
Mellor, A. L. and D. H. Munn (2004). “IDO expression by dendritic cells: Tolerance and tryptophan catabolism.” Nature Reviews Immunology 4(10): 762-774.
Munn, D. H. (2011). “Indoleamine 2,3-dioxygenase, Tregs and cancer.” Current Medicinal Chemistry 18(15): 2240-2246.
Munn, D. H., E. Shafizadeh, J. T. Attwood, I. Bondarev, A. Pashine and A. L. Mellor (1999). “Inhibition of T cell proliferation by macrophage tryptophan catabolism.” J Exp Med 189(9): 1363-1372.
Pilotte, L., P. Larrieu, V. Stroobant, D. Colau, E. Dolušić, R. Frédérick, E. De Plaen, C. Uyttenhove, J. Wouters, B. Masereel and B. J. Van Den Eynde (2012). “Reversal of tumoral immune resistance by inhibition of tryptophan 2,3-dioxygenase.” Proceedings of the National Academy of Sciences of the United States of America 109(7): 2497-2502.
Sekkai, D., O. Guittet, G. Lemaire, J. P. Tenu and M. Lepoivre (1997). “Inhibition of nitric oxide synthase expression and activity in macrophages by 3-hydroxyanthranilic acid, a tryptophan metabolite.” Arch Biochem Biophys 340(1): 117-123.
Suzuki, Y., T. Suda, K. Asada, S. Miwa, M. Suzuki, M. Fujie, K. Furuhashi, Y. Nakamura, N. Inui, T. Shirai, H. Hayakawa, H. Nakamura and K. Chida (2012). “Serum indoleamine 2,3-dioxygenase activity predicts prognosis of pulmonary tuberculosis.” Clin Vaccine Immunol 19(3): 436-442.
Tattevin, P., D. Monnier, O. Tribut, J. Dulong, N. Bescher, F. Mourcin, F. Uhel, Y. Le Tulzo and K. Tarte (2010). “Enhanced indoleamine 2,3-dioxygenase activity in patients with severe sepsis and septic shock.” J Infect Dis 201(6): 956-966.
Tenorio, A. R., Y. Zheng, R. J. Bosch, S. Krishnan, B. Rodriguez, P. W. Hunt, J. Plants, A. Seth, C. C. Wilson, S. G. Deeks, M. M. Lederman and A. L. Landay (2014). “Soluble markers of inflammation and coagulation but not T-cell activation predict non-AIDS-defining morbid events during suppressive antiretroviral treatment.” J Infect Dis 210(8): 1248-1259.
Wainwright, D. A., I. V. Balyasnikova, A. L. Chang, A. U. Ahmed, K.-S. Moon, B. Auffinger, A. L. Tobias, Y. Han and M. S. Lesniak (2012). “IDO Expression in Brain Tumors Increases the Recruitment of Regulatory T Cells and Negatively Impacts Survival.” Clinical Cancer Research 18(22): 6110-6121.
Wainwright, D. A., A. L. Chang, M. Dey, I. V. Balyasnikova, C. K. Kim, A. Tobias, Y. Cheng, J. W. Kim, J. Qiao, L. Zhang, Y. Han and M. S. Lesniak (2014). “Durable therapeutic efficacy utilizing combinatorial blockade against IDO, CTLA-4, and PD-L1 in mice with brain tumors.” Clinical Cancer Research 20(20): 5290-5301.
Yue, E. W., B. Douty, B. Wayland, M. Bower, X. Liu, L. Leffet, Q. Wang, K. J. Bowman, M. J. Hansbury, C. Liu, M. Wei, Y. Li, R. Wynn, T. C. Burn, H. K. Koblish, J. S. Fridman, B. Metcalf, P. A. Scherle and A. P. Combs (2009). “Discovery of potent competitive inhibitors of indoleamine 2,3-dioxygenase with in vivo pharmacodynamic activity and efficacy in a mouse melanoma model.” Journal of Medicinal Chemistry 52(23): 7364-7367.

### SUMMARY OF THE INVENTION

Briefly, in one aspect, the present invention discloses compounds of Formula I

[chemical structure]

or a pharmaceutically acceptable salt thereof wherein:

each X is CH or one X is N and the other two are CH;

R¹ and R² are independently H or C₁₋₃alkyl, or R¹ and R² may join together with the carbon atom to which they are bonded to form a 3-6 membered cycloalkyl;

R³ is CO₂H or an acid isostere;

R⁴ is a 5 or 6-membered heterocycle or heteroaryl containing 1 to 4 heteroatoms selected from N, S, and O, wherein said heterocycle or heteroaryl may optionally be substituted by 1 or 2 substituent selected from the group consisting of halogen, C₃₋₆cycloalkyl, CH₂OH, C(O)NH₂, CN, CH₂OC₁₋₃alkyl, C₁₋₃alkyl optionally substituted by 1-3 halogens, and wherein said CH₂OH is optionally converted into a prodrug by converting the CH₂OH group to a CH₂OC(O)CH₃, CH₂OC(O)C(C₁₋₄alkyl)₃, or OP(O)(OH)₂ group, or OP(O)(OC₁₋₄ alkyl)₂ group;

R⁵ is a 4, 5, or 6-membered cycloalkyl optionally substituted with an OH or a OCH₃ group or 1 or 2 halogens, or a 5 or 6-membered heterocycle containing an O or a N optionally substituted with a substituent selected from the group consisting of halogen, OH, C₁₋₄alkyl; OC₁₋₃alkyl, C(O)C₃₋₆cycloalkyl, BOC, C(O)C₁₋₃alkyl-O—C₁₋₃alkyl; C(O)C₁₋₃alkyl; C(O)—O—C₁₋₃ alkyl, and a 4 to 6-membered heterocycle or heteroaryl containing 1 to 4 heteroatoms selected from N, S, and O, wherein said heterocycle or heteroaryl may optionally be substituted by 1 substituent selected from the group consisting of halogen, C₃₋₆cycloalkyl, CH₂OH, C(O)NH₂, CN, CH₂OC₁₋₃alkyl, C₁₋₃alkyl optionally substituted by 1-3 halogens.

In another aspect, the present invention discloses a method for treating diseases or conditions that would benefit from inhibition of IDO.

In another aspect, the present invention discloses pharmaceutical compositions comprising a compound of Formula I or a pharmaceutically acceptable salt thereof.

In another aspect, the present invention provides a compound of Formula I or a pharmaceutically acceptable salt thereof for use in therapy.

In another aspect, the present invention provides a compound of Formula I or a pharmaceutically acceptable salt thereof for use in treating diseases or condition that would benefit from inhibition of IDO.

In another aspect, the present invention provides use of a compound of Formula I or a pharmaceutically acceptable salt thereof in the manufacture of a medicament for use in treating diseases or conditions that would benefit from inhibition of IDO.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

### DETAILED DESCRIPTION OF REPRESENTATIVE EMBODIMENTS

Preferably R¹ and R² are independently H or CH₃, or R¹ and R² together with the carbon to which they are bonded form a cyclopropyl ring.

Preferably R³ is CO₂H, —C(O)—NH—S(O)₂—CF₃, or —C(O)—NH—S(O)₂—CH₃.

Preferably R⁴ is a pyridine, thiadiazole, pyrimidine, pyrazine, pyridazine, triazol, or thiazol.

Preferably R⁴ is unsubstituted or substituted with 1 or 2 substituent selected from the group consisting of F, Cl, and cyclopropyl.

Preferably R⁵ is C₁₋₄alkyl or a 6-membered heterocycle containing an O or a N.

Preferably R⁵ is unsubstituted.

Examples of suitable acid isosteres, includes for example

[chemical structure]

wherein R¹ and R² in the above list of isosters are independently C₁₋₆ alkyl or C₁₋₆fluoroalkyl.

Preferred pharmaceutical composition include unit dosage forms. Preferred unit dosage forms include tablets.

In particular, it is expected that the compounds and composition of this invention will be useful for prevention and/or treatment of HIV; including the prevention of the progression of AIDS and general immunosuppression. It is expected that in many cases such prevention and/or treatment will involve treating with the compounds of this invention in combination with at least one other drug thought to be useful for such prevention and/or treatment. For example, the IDO inhibitors of this invention may be used in combination with other immune therapies such as immune checkpoints (PD1, CTLA4, ICOS, etc.) and possibly in combination with growth factors or cytokine therapies (IL21, IL-7, etc.).

In is common practice in treatment of HIV to employ more than one effective agent. Therefore, in accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCRS inhibitors. Examples of such additiona agents are Dolutegravir, Bictegravir. and Cabotegravir.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or ACN are preferred.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the pharmaceutical formulation containing a compound of Formula I or a salt thereof is a formulation adapted for oral or parenteral administration. In another embodiment, the formulation is a long-acting parenteral formulation. In a further embodiment, the formulation is a nano-particle formulation.

The present invention is directed to compounds, compositions and pharmaceutical compositions that have utility as novel treatments for immunosuppresion. While not wanting to be bound by any particular theory, it is thought that the present compounds are able to inhibit the enzyme that catalyzes the oxidative pyrrole ring cleavage reaction of I-Trp to N-formylkynurenine utilizing molecular oxygen or reactive oxygen species.

Therefore, in another embodiment of the present invention, there is provided a method for the prevention and/or treatment of HIV; including the prevention of the progression of AIDS and general immunosuppression.

### EXAMPLES

The following examples serve to more fully describe the manner of making and using the above-described invention. It is understood that these examples in no way serve to limit the true scope of the invention, but rather are presented for illustrative purposes. In the examples and the synthetic schemes below, the following abbreviations have the following meanings. If an abbreviation is not defined, it has its generally accepted meaning.
ACN=Acetonitrile
AIBN=azobisisobutyronitrile
aq.=Aqueous
μL or μL=Microliters
μM or μM=Micromolar
NMR=nuclear magnetic resonance
boc=tert-butoxycarbonyl
br=Broad
Cbz=Benzyloxycarbonyl
CDl=1,1′-carbonyldiimidazole
d=Doublet
δ=chemical shift
° C.=degrees celcius
DCM=Dichloromethane
dd=doublet of doublets
DHP=Dihydropyran
DIAD=diisopropyl azodicarboxylate
DIEA or DIPEA=N,N-diisopropylethylamine
DMAP=4-(dimethylamino)pyridine
DMEM=Dulbeco's Modified Eagle's Medium
EtOAc=ethyl acetate
h or hr=Hours
HATU=1-[Bis(dimethylamino)methylene]-1H-1,2,3-triazolo[4,5-b]pyridinium 3-oxid hexafluorophosphate
HCV=hepatitis C virus
HPLC=high performance liquid chromatography
Hz=Hertz
IU=International Units
IC₅₀=inhibitory concentration at 50% inhibition
J=coupling constant (given in Hz unless otherwise indicated)
LCMS=liquid chromatography-mass spectrometry
m=Multiplet
M=Molar
M+H⁺=parent mass spectrum peak plus H⁺
MeOH=Methanol
mg=Milligram
min=Minutes
mL=Milliliter
mM=Millimolar
mmol=Millimole
MS=mass spectrum
MTBE=methyl tert-butyl ether
N=Normal
NFK=N-formylkynurenine
NBS=N-bromosuccinimide
nm=Nanomolar
PE=petroleum ether
ppm=parts per million
q.s.=sufficient amount
s=Singlet
RT=room temperature
Rf=retardation factor
sat.=Saturated
t=Triplet
TEA=Triethylamine
TFA=trifluoroacetic acid
TFAA=trifluoroacetic anhydride
THF=Tetrahydrofuran

### Equipment Description

¹H NMR spectra were recorded on a Bruker Ascend 400 spectrometer or a Varian 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters ACQUITY UPLC with SQ Detectors using a Waters BEH C18, 2.1×50 mm, 1.7 μm using a gradient elution method.

Solvent A: 0.1% formic acid (FA) in water;

Solvent B: 0.1% FA in acetonitrile;

30% B for 0.5 min followed by 30-100% B over 2.5 min.

[chemical structure]

[chemical structure]

### Preparation of methyl 2-(4-fluorophenyl)acetate

[chemical structure]

A mixture of 2-(4-fluorophenyl)acetic acid (10.0 g, 64.9 mmol) and concentrated H₂SO₄ (1.0 mL) in MeOH (100 mL) was heated at reflux temperature overnight. The solvent was removed by evaporation in vacuum. The residue was diluted with water and extracted with EtOAc. The organic layers were combined and washed sequentially with sat. aqueous NaHCO₃, water, and brine, and dried over MgSO₄. Filtration and concentration in vacuum gave the title compound (11.2 g, quantitative) as pale oil, which was used in the following step without purification. (ESI) m/z calcd for C₉H₉FO₂: 168.06. Found: 169.16 (M+1)⁺.

### Preparation of methyl 2-(4-fluorophenyl)-2-methylpropanoate

[chemical structure]

At 0° C., to a suspension of NaH (6.7 g, 167.7 mmol) in THF (100 mL), a solution of methyl 2-(4-fluorophenyl)acetate (9.4 g, 55.9 mmol) and iodidemethane (23.8 g, 167.7 mmol) in THF (50 mL) was added drop wise. The resulting mixture was allowed to warm up to room temperature and stirred overnight. The residue was quenched with saturated aq. NH₄Cl and extracted with EtOAc. The organics were washed sequentially with water and brine, and dried over Na₂SO₄. Filtration and concentration in vacuum gave a crude product, which was purified by flash chromatography (silica gel, 0-30% EtOAc in PE) to afford the title compound (7.6 g, 69% yield). (ESI) m/z calcd for C₁₃H₁₃FO₂: 196.09. Found: 197.17 (M+1)⁺.

### Preparation of methyl 2-(4-fluoro-3-nitrophenyl)-2-methylpropanoate

[chemical structure]

At 0° C., to conc. sulfuric acid (11 mL) was added methyl 2-(4-fluorophenyl)-2-methylpropanoate (7.6 g, 38.8 mmol) in one portion, followed by adding KNO₃ (3.8 g, 38.8 mmol) portion wise. After stirred at 0° C. for 3 h, the reaction mixture was poured into ice-water and extracted with EtOAc. The organic layer was washed with brine and dried over Na₂SO₄. Solvent was removed under vacuum and the residue was purified by flash chromatography (silica gel, 0-50% ethyl acetate in petroleum ether) to afford the title compound (7.6 g, 81%) as a yellow oil. (ESI) m/z calcd for C₁₁H₁₂FNO₄: 241.08. Found: 242.20 (M+1)⁺.

### Preparation of methyl 2-(4-(isobutyl(tetrahydro-2H-pyran-4-yl)amino)-3-nitrophenyl)-2-methylpropanoate

[chemical structure]

A mixture of methyl methyl 2-(4-fluoro-3-nitrophenyl)-2-methylpropanoate (7.2 g, 30.0 mmol) and N-isobutyltetrahydro-2H-pyran-4-amine (11.8 g, 75 mmol) was stirred at 160° C. under N₂ atmosphere for 7 hr. The reaction mixture was purified by column chromatography (silica gel, 0-40% EtOAc in PE) to afford the title compound (4.7 g, 42% yield) as a red oil. (ESI) m/z calcd for C₂₀H₃₀N₂O₅: 378.22. Found: 379.42 (M+1)⁺.

### Preparation of methyl 2-(3-amino-4-(isobutyl(tetrahydro-2H-pyran-4-yl)amino)phenyl)-2-methylpropanoate

[chemical structure]

A mixture of methyl 2-(4-(isobutyl(tetrahydro-2H-pyran-4-yl)amino)-3-nitrophenyl)-2-methylpropanoate (4.7 g, 12.4 mmol) and 10% Pd/C (1.41 g) in EtOAc (50 mL) was stirred at room temperature under H₂ atmosphere (15 psi) overnight. The resulting mixture was filtered through a pad of Celite and the filtrate was concentrated under reduced pressure to give the crude product which was purified by flash chromatography (silica gel, 0-50% EtOAc in PE) to afford the title compound (4.2 g, 96% yield) as a brown oil. (ESI) m/z calcd for C₂₀H₃₂N₂O₃: 348.24. Found: 349.36 (M+1)⁺.

### Preparation of methyl 2-(3-(5-chloropyridin-2-yl)amino)-4-(isobutyl(tetrahydro-2H-pyran-4-yl)amino)phenyl)-2-methylpropanoate

[chemical structure]

A mixture of methyl 2-(3-amino-4-(isobutyl(tetrahydro-2H-pyran-4-yl)amino)phenyl)-2-methylpropanoate (550 mg, 1.59 mmol), 2-bromo-5-chloropyridine (460 mg, 2.39 mmol), Pd₂(dba)₃ (146 mg, 0.159 mmol), Xantphos (185 mg, 0.318 mmol) and Cs₂CO₃ (1.04 g, 3.18 mmol) in dioxane (12 mL) was stirred at 100° C. under N₂ atmosphere overnight. The resulting mixture was partitioned between EtOAc and H₂O.

The organic layer was washed with brine, dried over Na₂SO₄, filtered and concentrated to give the crude product which was purified by flash chromatography (silica gel, 0-50% EtOAc in PE) to afford the title compound (650 mg, 89% yield). LCMS (ESI) m/z calcd for C₂₅H₃₄ClN₃O₃: 459.23. Found: 460.05/462.42 (M/M+2)⁺.

### Example 1

### Preparation of 2-(3-((5-chloropyridin-2-yl)amino)-4-(isobutyl(tetrahydro-2H-pyran-4-yl)amino)phenyl)-2-methylpropanoic acid

[chemical structure]

To a solution of methyl 2-(3-((5-chloropyridin-2-yl)amino)-4-(isobutyl(tetrahydro-2H-pyran-4-yl)amino)phenyl)-2-methylpropanoate (150 mg, 0.33 mmol) in MeOH (3 mL) was added 4N NaOH aq. (0.5 mL). After stirred at 70° C. for 4 h, the resulting mixture was neutralized with 1N HCl and extracted with EtOAc. The organic layer was washed with brine, dried over Na₂SO₄, filtered and concentrated to give the crude product which was purified by HPLC (C18, 10-70% MeCN in H₂O with 0.1% formic acid) to afford the title compound (78 mg, 54% yield) as a white powder. ¹H NMR (400 MHz, DMSO) δ 12.33 (s, 1H), 8.23 (d, J=2.0 Hz, 1H), 8.20-8.14 (m, 2H), 7.69-7.63 (m, 1H), 7.20 (d, J=8.3 Hz, 1H), 7.00 (d, J=8.9 Hz, 1H), 6.96-6.90 (m, 1H), 3.85-3.77 (m, 2H), 3.14 (t, J=11.2 Hz, 2H), 2.89-2.82 (m, 1H), 2.82-2.77 (m, 2H), 1.70-1.62 (m, 2H), 1.57-1.49 (m, 2H), 1.47 (s, 6H), 1.37-1.30 (m, 1H), 0.82 (d, J=6.6 Hz, 6H). LCMS (ESI) m/z calcd for C₂₄H₃₂ClN₃O₃: 445.21. Found: 446.38/448.30 (M/M+2)⁺.

### Example 2

### Preparation of 2-(3-((5-chloropyridin-2-yl)amino)-4-(isobutyl(tetrahydro-2H-pyran-4-yl)amino)phenyl)-2-methyl-N-(methylsulfonyl)propanamide

[chemical structure]

To a solution of 2-(3-((5-chloropyridin-2-yl)amino)-4-(isobutyl(tetrahydro-2H-pyran-4-yl)amino)phenyl)-2-methylpropanoic acid (150 mg, 0.34 mmol), methanesulfonamide (36 mg, 0.38 mmol) and DMAP (9 mg, 0.07 mmol) in DMF (3 mL), was added DCC (85 mg, 0.41 mmol) in one portion. After stirred at room temperature for 5 h, the resulting mixture was partitioned between EtOAc and H₂O. The organic layer was washed with brine, dried over Na₂SO₄, filtered and concentrated to give the crude product which was purified by HPLC (C18, 10-80% MeCN in H₂O with 0.1% formic acid) to afford the title compound (22 mg, 13% yield) as a white powder. ¹H NMR (400 MHz, DMSO) δ 11.33 (s, 1H), 8.20-8.13 (m, 2H), 8.08 (s, J=1.6 Hz, 1H), 7.66 (dd, J=8.9, 2.7 Hz, 1H), 7.24 (d, J=8.3 Hz, 1H), 7.05 (d, J=8.9 Hz, 1H), 6.87 (dd, J=8.3, 2.1 Hz, 1H), 3.84-3.77 (m, 2H), 3.25-3.08 (m, 5H), 2.87-2.78 (m, 3H), 1.70-1.63 (m, 2H), 1.57-1.42 (m, 8H), 1.38-1.32 (m, 1H), 0.83 (d, J=6.6 Hz, 6H). LCMS (ESI) m/z calcd for C₂₅H₃₅ClN₄O₄S: 522.21. Found: 523.45/525.62 (M/M+2)⁺.

### Example 3

### Preparation of 2-(3-((5-chloropyridin-2-yl)amino)-4-(isobutyl(tetrahydro-2H-pyran-4-yl)amino)phenyl)-2-methyl-N-((trifluoromethyl)sulfonyl)propanamide

[chemical structure]

To a solution of 2-(3-((5-chloropyridin-2-yl)amino)-4-(isobutyl(tetrahydro-2H-pyran-4-yl)amino)phenyl)-2-methylpropanoic acid (150 mg, 0.34 mmol), trifluoromethanesulfonamide (57 mg, 0.38 mmol) and DMAP (9 mg, 0.07 mmol) in DMF (3 mL), was added DCC (85 mg, 0.41 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-70% MeCN in H₂O with 0.1% formic acid) to afford the title compound (21 mg, 11% yield) as a white powder. ¹H NMR (400 MHz, DMSO) δ 8.30 (s, 1H), 8.19 (d, J=2.5 Hz, 1H), 7.87 (s, 1H), 7.70-7.63 (m, 1H), 7.23 (d, J=8.4 Hz, 1H), 7.04 (d, J=9.0 Hz, 1H), 6.99 (d, J=6.7 Hz, 1H), 3.87-3.76 (m, 2H), 3.13 (t, J=11.2 Hz, 2H), 3.01-2.87 (m, 3H), 1.69-1.61 (m, 2H), 1.56-1.48 (m, 2H), 1.39 (s, J=11.5 Hz, 6H), 1.29-1.24 (m, 1H), 0.80 (d, J=6.6 Hz, 6H). The proton of sulfonamide group was not observed. LCMS (ESI) m/z calcd for C₂₅H₃₂ClF₃N₄O₄S: 576.18. Found: 577.63/579.64 (M/M+2)⁺.

[chemical structure]

### Preparation of methyl 2-(4-(diisobutylamino)-3-nitrophenyl)-2-methylpropanoate

[chemical structure]

A mixture of methyl methyl 2-(4-fluoro-3-nitrophenyl)-2-methylpropanoate (1.0 g, 4.0 mmol), diisobutylamine (2.2 mL, 12.3 mmol), DIPEA (3.6 mL, 20.5 mmol) and NMP (10 mL) was stirred at 110° C. under N₂ atmosphere for 17 hr. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (800 mg, 57% yield)I. (ESI) m/z calcd for C₁₉H₃₀N₂O₄: 350.22. Found: 351.63 (M+1)⁺.

### Preparation of methyl 2-(3-amino-4-(diisobutylamino)phenyl)-2-methylpropanoate

[chemical structure]

A mixture of methyl 2-(4-(diisobutylamino)-3-nitrophenyl)-2-methylpropanoate (800 mg, 2.28 mmol) and 10% Pd/C (120 mg) in EtOAc (50 mL) was stirred at 50° C. under H₂ atmosphere (15 psi) overnight. The resulting mixture was filtered through a pad of Celite and the filtrate was concentrated under reduced pressure to give the crude product which was purified by flash chromatography (silica gel, 0-50% EtOAc in PE) to afford the title compound (680 mg, 93% yield). (ESI) m/z calcd for C₁₉H₃₂N₂O₂: 320.25. Found: 321.67 (M+1)⁺.

### Preparation of methyl 2-(3-((5-chloropyridin-2-yl)amino)-4-(diisobutylamino)phenyl)-2-methylpropanoate

[chemical structure]

A mixture of methyl 2-(3-amino-4-(diisobutylamino)phenyl)-2-methylpropanoate (250 mg, 0.78 mmol), 2-bromo-5-chloropyridine (301 mg, 1.56 mmol), Pd₂(dba)₃ (71 mg, 0.156 mmol), Xantphos (90 mg, 0.156 mmol) and Cs₂CO₃ (588 mg, 1.56 mmol) in toluene (10 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (180 mg, 53% yield). LCMS (ESI) m/z calcd for C₂₄H₃₄ClN₃O₂: 431.23. Found: 432.64/434.61 (M/M+2)⁺.

### Example 12

### Preparation of 2-(3-((5-chloropyridin-2-yl)amino)-4-(diisobutylamino)phenyl)-2-methylpropanoic acid

[chemical structure]

To a solution of methyl 2-(3-((5-chloropyridin-2-yl)amino)-4-(diisobutylamino)phenyl)-2-methylpropanoate (180 mg, 0.42 mmol) in MeOH (6 mL) was added 1 N NaOH aq. (5 mL). After stirred at room temperature overnight, the resulting mixture was neutralized with 1N HCl and extracted with EtOAc. The organic layer was washed with brine, dried over Na₂SO₄, filtered and concentrated to give the crude product which was purified by HPLC (C18, 10-60% MeCN in H₂O with 0.1% formic acid) to afford the title compound (78 mg, 54% yield) as a white powder. U26886-086-1 ¹H NMR (400 MHz, DMSO) δ 12.15 (br, 1H), 8.27-8.12 (m, 3H), 7.68 (dd, J=8.9, 2.7 Hz, 1H), 7.22 (d, J=8.4 Hz, 1H), 6.94 (dd, J=8.3, 2.3 Hz, 1H), 6.82 (d, J=8.9 Hz, 1H), 2.60 (d, J=7.1 Hz, 4H), 1.70-1.59 (m, 2H), 1.47 (s, 6H), 0.86 (d, J=6.6 Hz, 12H). LCMS (ESI) m/z calcd for C₂₃H₃₂ClN₃O₂: 417.22. Found: 418.73/420.71 (M/M+2)⁺.

[chemical structure]

### Preparation of methyl 2-(4-(diisobutylamino)-3-((3-(trifluoromethyl)-1,2,4-thiadiazol-5-yl)amino)phenyl)-2-methylpropanoate

[chemical structure]

A mixture of methyl 2-(3-amino-4-(diisobutylamino)phenyl)-2-methylpropanoate (200 mg, 0.64 mmol) and 5-chloro-3-(trifluoromethyl)-1,2,4-thiadiazole (180 mg, 0.96 mmol) in MeCN (4 mL) was stirred at 90° C. under N₂ atmosphere overnight. The resulting mixture was partitioned between EtOAc and H₂O. The layers were separated and the organic layer was washed with brine, dried over Na₂SO₄, filtered and concentrated to give the crude product which was purified by flash chromatography (silica gel, 0-60% EtOAc in PE) to afford the title compound (150 mg, 51% yield). LCMS (ESI) m/z calcd for C₂₂H₃₁F₃N₄O₂S: 472.21. Found: 473.61 (M+1)⁺.

### Example 13

### Preparation of 2-(4-(diisobutylamino)-3-((3-(trifluoromethyl)-1,2,4-thiadiazol-5-yl)amino)phenyl)-2-methylpropanoic acid

[chemical structure]

A solution of methyl 2-(4-(diisobutylamino)-3-((3-(trifluoromethyl)-1,2,4-thiadiazol-5-yl)amino)phenyl)-2-methylpropanoate (150 mg, 0.32 mmol) in MeOH (6 mL) and 1N NaOH aq. solution (5 mL) was stirred at room temperature for overnight. The resulting mixture was neutralized with 1N HCl aq. solution and extracted with EtOAc. The organic layer was washed with brine, dried over Na₂SO₄, filtered and concentrated to give the crude product which was purified by HPLC (C18, 10-100% MeCN in H₂O with 0.1% formic acid) to afford the title compound (103 mg, 70% yield) as a white powder. ¹H NMR (400 MHz, DMSO) δ 12.21 (br, 1H), 10.24 (s, 1H), 7.79 (s, 1H), 7.28-7.06 (m, 2H), 2.77 (d, J=7.0 Hz, 4H), 1.80-1.59 (m, 2H), 1.46 (s, 6H), 0.79 (d, J=6.6 Hz, 12H). LCMS (ESI) m/z calcd for C₂₁H₂₉F₃N₁₄O₂S: 458.20. Found: 459.59 (M+1)⁺.

[chemical structure]

[chemical structure]

### Preparation of 1-(4-fluorophenyl)cyclopropane-1-carbonitrile

[chemical structure]

To a mixture of 1-(4-fluorophenyl)acetonitrile (20.3 g, 150 mmol), 1-bromo-2-chloroethane (25 mL, 300 mmol) and benzyltriethylammonium chloride (683 mg, 3.00 mmol) was added 50% aqueous NaOH (84 g, 1.05 mol), and the resulting mixture was heated at 50° C. overnight. After cooling, the mixture was poured into water and extracted with diisopropyl ether. The organic layer was washed sequentially with water, 1 N aqueous HCl, and brine, and dried over MgSO₄. Filtration, concentration in vacuum afforded the title compound (16.4 g 68%) as a yellow oil, which was used in the following step without further purification. (ESI) m/z calcd for C₁₀H₈FN: 161.06. Found: 162.28 (M+1)⁺.

### Preparation of 1-(4-fluorophenyl)cyclopropane-1-carboxamide

[chemical structure]

To a solution of 1-(4-fluorophenyl)cyclopropane-1-carbonitrile (16.4 g, 102 mmol) in acetone (140 mL) was added 4 N aqueous NaOH (100 mL) at room temperature. 30% H₂O₂ (150 mL) was added dropwise to the solution with cooling in an ice-water bath. The mixture was allowed to stand at room temperature and stirred for an additional 2 h. The reaction mixture was cooled in an ice-water bath, and aqueous Na₂SO₃ (10% in water, 159 mmol) was added to the mixture. The solvent was removed by evaporation in vacuum, and the precipitated solid was collected by filtration and washed with water and n-hexane to give the title compound (17.0 g, 93%) as a white solid. (ESI) m/z calcd for C₁₀H₁₀FNO: 179.07. Found: 180.11 (M+1)⁺.

### Preparation of 1-(4-fluorophenyl)cyclopropane-1-carboxylic acid

[chemical structure]

A mixture of 1-(4-fluorophenyl)cyclopropane-1-carboxamide (17.0 g, 94.8 mmol) in 6 N aqueous HCl (95 mL) and 1,4-dioxane (150 mL) was heated at reflux temperature overnight. The solvent was removed by evaporation in vacuum, and the residue extracted with EtOAc. The organic layer was washed with brine and dried over MgSO₄. Filtration and concentration in vacuum gave the title compound (16.8 g, 98%) as a white solid. (ESI) m/z calcd for C₁₀H₉FO₂: 180.06. Found: 181.12 (M+1)⁺.

### Preparation of methyl 1-(4-fluorophenyl)cyclopropane-1-carboxylate

[chemical structure]

A mixture of 1-(4-fluorophenyl)cyclopropane-1-carboxylic acid (11.8 g, 65.5 mmol) and concentrated H₂SO₄ (1.5 mL) in MeOH (100 mL) was heated at reflux temperature for 8 h. The solvent was removed by evaporation in vacuum. The residue was diluted with water and extracted with EtOAc. The organics were washed sequentially with sat. aqueous NaHCO₃, water, and brine, and dried over MgSO₄. Filtration and concentration in vacuum gave the title compound (12.7 g, quantitative) as yellow oil, which was used in the following step without purification. (ESI) m/z calcd for C₁₁H₁₁FO₂: 194.07. Found: 195.31 (M+1)⁺.

### Preparation of methyl 1-(4-fluoro-3-nitrophenyl)cyclopropane-1-carboxylate

[chemical structure]

At 0° C., to conc. sulfuric acid (8 mL) was added methyl 1-(4-fluorophenyl)cyclopropane-1-carboxylate (5.6 g, 28.8 mmol) in one portion, followed by adding KNO₃ (2.9 g, 28.8 mmol) portion wise. After stirred at 0° C. for 3 h, the reaction mixture was poured into ice-water and extracted with EtOAc. The organic layer was washed with brine and dried over Na₂SO₄. Solvent was removed under vacuum and the residue was purified by flash chromatography (silica gel, 0-50% ethyl acetate in petroleum ether) to afford the title compound (5.7 g, 60%) as yellow oil. (ESI) m/z calcd for C₁₁H₁₀FNO₄: 239.06. Found: 240.14 (M+1)⁺.

### Preparation of methyl 1-(4-(isobutyl(tetrahydro-2H-pyran-4-yl)amino)-3-nitrophenyl)cyclopropane-1-carboxylate

[chemical structure]

A mixture of methyl 1-(4-fluoro-3-nitrophenyl)cyclopropane-1-carboxylate (5.7 g, 23.8 mmol) and N-isobutyltetra hydro-2H-pyran-4-amine (11.3 g, 71.5 mmol) was stirred at 160° C. under N₂ atmosphere for 7 hr. The reaction mixture was purified by column chromatography (silica gel, 0-10% EtOAc in PE) to afford the title compound (3.4 g, 40% yield) as a red oil. LCMS (ESI) m/z calcd for C₂₀H₂₈N₂O₅: 376.20. Found: 377.32 (M+1)⁺.

### Preparation of methyl 1-(3-amino-4-(isobutyl(tetrahydro-2H-pyran-4-yl)amino)phenyl)cyclopropane-1-carboxylate

[chemical structure]

A mixture of methyl 1-(4-(isobutyl(tetrahydro-2H-pyran-4-yl)amino)-3-nitrophenyl)cyclopropane-1-carboxylate (3.1 g, 8.24 mmol) and 10% Pd/C (1.1 g) in EtOAc (30 mL) was stirred at room temperature under H₂ atmosphere (15 psi) for 6 h. The resulting mixture was filtered through a pad of Celite and the filtrate was concentrated under reduced pressure to give the crude product which was purified by flash chromatography (silica gel, 0-20% EtOAc in PE) to afford the title compound (2.1 g, 81% yield) as a yellow oil. LCMS (ESI) m/z calcd for C₂₀H₃₀N₂O₃: 346.23. Found: 347.33 (M+1)⁺.

### Preparation of methyl 1-(3-((5-chloropyridin-2-yl)amino)-4-(isobutyl(tetrahydro-2H-pyran-4-yl)amino)phenyl)cyclopropane-1-carboxylate

[chemical structure]

A mixture of methyl 1-(3-amino-4-(isobutyl(tetrahydro-2H-pyran-4-yl)amino)phenyl)cyclopropane-1-carboxylate (550 mg, 1.59 mmol), 2-bromo-5-chloropyridine (460 mg, 2.39 mmol), Pd₂(dba)₃ (146 mg, 0.159 mmol), Xantphos (185 mg, 0.318 mmol) and Cs₂CO₃ (1.04 g, 3.18 mmol) in dioxane (12 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (566 mg, 71% yield). LCMS (ESI) m/z calcd for C₂₅H₃₂ClN₃O₃: 457.21. Found: 458.33/460.26 (M/M+2)⁺.

### Example 5

### Preparation of 1-(3-((5-chloropyridin-2-yl)amino)-4-(isobutyl(tetrahydro-2H-pyran-4-yl)amino)phenyl)cyclopropane-1-carboxylic acid

[chemical structure]

To a solution of methyl 1-(3-amino-4-(isobutyl(tetrahydro-2H-pyran-4-yl)amino)phenyl)cyclopropane-1-carboxylate (566 mg, 1.24 mmol) in MeOH (3 mL) was added 4N NaOH aq. (0.5 mL). After stirred at 25° C. for 4 h, the resulting mixture was neutralized with 1N HCl and extracted with EtOAc. The organic layer was washed with brine, dried over Na₂SO₄, filtered and concentrated to give the crude product which was purified by HPLC (C18, 10-100% MeCN in H₂O with 0.1% formic acid) to afford the title compound (523 mg, 95% yield) as a pale powder. ¹H NMR (400 MHz, DMSO) δ 12.19 (br, 1H), 8.24-8.20 (m, 2H), 8.16 (d, J=1.9 Hz, 1H), 7.67 (dd, J=8.9, 2.6 Hz, 1H), 7.18 (d, J=8.2 Hz, 1H), 7.01 (d, J=8.9 Hz, 1H), 6.90 (dd, J=8.1, 1.9 Hz, 1H), 3.87-3.76 (m, 2H), 3.14 (t, J=11.3 Hz, 2H), 2.87-2.77 (m, 3H), 1.71-1.62 (m, J=11.0 Hz, 2H), 1.58-1.47 (m, 2H), 1.43 (dd, J=6.4, 3.7 Hz, 2H), 1.38-1.30 (m, 1H), 1.16-1.10 (m, 2H), 0.83 (d, J=6.6 Hz, 6H). LCMS (ESI) m/z calcd for C₂₄H₃₀ClN₃O₃: 443.20. Found: 444.30/446.28 (M/M+2)⁺.

### Example 4

### Preparation of 1-(3-((5-chloropyridin-2-yl)amino)-4-(isobutyl(tetrahydro-2H-pyran-4-yl)amino)phenyl)-N-(methylsulfonyl)cyclopropane-1-carboxamide

[chemical structure]

To a solution of 1-(3-((5-chloropyridin-2-yl)amino)-4-(isobutyl(tetrahydro-2H-pyran-4-yl)amino)phenyl)cyclopropane-1-carboxylic acid (150 mg, 0.34 mmol), methanesulfonamide (36 mg, 0.38 mmol) and DMAP (9 mg, 0.07 mmol) in DCM (3 mL), was added DCC (85 mg, 0.41 mmol) in one portion. After stirred at room temperature for 5 h, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (56 mg, 32% yield) as a white powder. ¹H NMR (400 MHz, DMSO) δ 11.08 (s, 1H), 8.30-8.08 (m, 3H), 7.67 (dd, J=8.8, 2.4 Hz, 1H), 7.22 (d, J=8.2 Hz, 1H), 7.06 (d, J=8.9 Hz, 1H), 6.86 (d, J=8.0 Hz, 1H), 3.82 (d, J=8.4 Hz, 2H), 3.32 (s, 3H), 3.14 (t, J=11.3 Hz, 2H), 2.91-2.74 (m, 3H), 1.74-1.61 (m, 2H), 1.52 (d, J=8.5 Hz, 1H), 1.49-1.42 (m, 2H), 1.30-1.20 (m, 2H), 1.19-1.07 (m, 2H), 0.83 (d, J=6.5 Hz, 6H). LCMS (ESI) m/z calcd for C₂₅H₃₃ClN₄O₄S: 520.19. Found: 521.30/523.27 (M/M+2)⁺.

[chemical structure]

[chemical structure]

### Preparation of methyl 1-(3-(2-(cyclopropanecarbonyl)hydrazine-1-carbothioamido)-4-(isobutyl(tetrahydro-2H-pyran-4-yl)amino)phenyl)cyclopropane-1-carboxylate

[chemical structure]

To a solution of methyl 1-(3-amino-4-(isobutyl(tetrahydro-2H-pyran-4-yl)amino)phenyl)cyclopropane-1-carboxylate (500 mg, 1.45 mmol) in MeCN (5 mL) was added TCDI (517 mg, 2.9 mmol) and the resulting reaction mixture was stirred at 25° C. under N₂ atmosphere for 3 hr. The resulting mixture was concentrated to give the crude isothiocyanate intermediate which was dissolved in EtOH (10 mL) and treated with cyclopropanecarbo hydrazide (218 mg, 2.18 mmol). After stirred at 50° C. overnight, the reaction mixture was concentrated to give the crude product, which was purified by flash chromatography (silica gel, 0-60% EtOAc in PE) to afford the title compound (734 mg, 100% yield) as a white solid. LCMS (ESI) m/z calcd for C₂₅H₃₆N₄O₄S: 488.25. Found: 489.35 (M+1)⁺.

### Preparation of methyl 1-(3-((5-cyclopropyl-1,3,4-thiadiazol-2-yl)amino)-4-(isobutyl(tetrahydro-2H-pyran-4-yl)amino)phenyl)cyclopropane-1-carboxylate

[chemical structure]

Methyl 1-(3-(2-(cyclopropanecarbonyl)hydrazine-1-carbothioamido)-4-(isobutyl (tetrahydro-2H-pyran-4-yl)amino)phenyl)cyclopropane-1-carboxylate (734 mg, 1.50 mmol) was added portion wise to conc. H₂SO₄ (10 mL) at 0° C. After stirred at room temperature for 3 hr, the mixture was carefully neutralized with aq. NaOH solution (4 N) to pH 5-6 and extracted with DCM. The combined organic layers were dried over Na₂SO₄ and concentrated to give the crude product (639 mg, 90% yield). which was used in the next step without purification. LCMS (ESI) m/z calcd for C₂₅H₃₄N₄O₃S: 470.24. Found: 471.73 (M+1)⁺.

### Example 14

### Preparation of 1-(3-((5-cyclopropyl-1,3,4-thiadiazol-2-yl)amino)-4-(isobutyl(tetrahydro-2H-pyran-4-yl)amino)phenyl)cyclopropane-1-carboxylic acid

[chemical structure]

To a solution of methyl 1-(3-((5-cyclopropyl-1,3,4-thiadiazol-2-yl)amino)-4-(isobutyl(tetrahydro-2H-pyran-4-yl)amino)phenyl)cyclopropane-1-carboxylate (639 mg, 1.36 mmol) in MeOH (3 mL) was added 4 N aq. NaOH (1 mL). After stirred at Et. for 5 hr, the resulting mixture was neutralized with 1N HCl and extracted with EtOAc. The organic layer was washed with brine, dried over Na₂SO₄, filtered and concentrated to give the crude product which was purified by HPLC (C18, 10-70% MeCN in H₂O with 0.1% formic acid) to afford the title compound (44 mg, 62% yield) as a as a pale powder. ¹H NMR (400 MHz, DMSO) δ 12.23 (br, 1H), 8.96 (s, 1H), 8.10 (d, J=1.9 Hz, 1H), 7.18 (d, J=8.2 Hz, 1H), 6.94 (dd, J=8.1, 2.0 Hz, 1H), 3.82 (dd, J=11.1, 3.5 Hz, 2H), 3.17 (t, J=11.3 Hz, 2H), 2.88 (ddd, J=11.4, 7.8, 3.8 Hz, 1H), 2.78 (d, J=6.7 Hz, 2H), 2.34-2.27 (m, 1H), 1.72-1.64 (m, 2H), 1.53-1.42 (m, 4H), 1.32 (dt, J=13.2, 6.6 Hz, 1H), 1.14-1.06 (m, 4H), 0.95-0.91 (m, 2H), 0.81 (d, J=6.6 Hz, 6H). LCMS (ESI) m/z calcd for C₂₄H₃₂N₄O₃S: 456.22. Found: 457.32 (M+1)⁺.

### Example 19

### Preparation of 1-(3-((5-cyclopropyl-1,3,4-thiadiazol-2-yl)amino)-4-(isobutyl(tetrahydro-2H-pyran-4-yl)amino)phenyl)-N-(methylsulfonyl)cyclopropane-1-carboxamide

[chemical structure]

To a solution of 1-(3-((5-cyclopropyl-1,3,4-thiadiazol-2-yl)amino)-4-(isobutyl(tetrahydro-2H-pyran-4-yl)amino)phenyl)cyclopropane-1-carboxylic acid (150 mg, 0.33 mmol), methanesulfonamide (35 mg, 0.36 mmol) and DMAP (9 mg, 0.07 mmol) in DCM (1 mL) and DMF (1 mL), was added DCC (83 mg, 0.40 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 20-100% MeCN in H₂O with 0.1% formic acid) to afford the title compound (30 mg, 17% yield) as a white powder. ¹H NMR (400 MHz, DMSO) δ 11.12 (s, 1H), 8.98 (s, 1H), 8.05 (s, 1H), 7.22 (d, J=8.2 Hz, 1H), 6.91 (dd, J=8.2, 2.1 Hz, 1H), 3.82 (dd, J=11.1, 3.4 Hz, 2H), 3.26-3.07 (m, 5H), 2.92-2.84 (m, 1H), 2.78 (d, J=6.8 Hz, 2H), 2.34-2.27 (m, 1H), 1.69 (d, J=10.8 Hz, 2H), 1.55-1.42 (m, 4H), 1.28-1.19 (m, 1H), 1.16-1.03 (m, 4H), 0.96-0.89 (m, 2H), 0.81 (d, J=6.6 Hz, 6H). LCMS (ESI) m/z calcd for C₂₅H₃₅N₅O₄S₂: 533.21. Found: 534.28 (M+1)⁺.

[chemical structure]

[chemical structure]

### Preparation of 5-bromo-N-isobutyl-3-nitro-N-(tetrahydro-2H-pyran-4-yl)pyridin-2-amine

[chemical structure]

A mixture of 5-bromo-2-chloro-3-nitropyridine (15.3 g, 64.5 mmol), N-isobutyltetrahydro-2H-pyran-4-amine (15.2 g, 96.7 mmol) and DIPEA (22.5 mL, 129 mmol) in NMP (150 mL) was stirred at 140° C. for 4 hr. The resulting mixture was partitioned between EtOAc and H₂O. The layers were separated and the organic layer was washed with brine, dried over Na₂SO₄, filtered and concentrated to give the crude product which was purified by flash chromatography (silica gel, 0-10% EtOAc in PE) to afford the title compound (9.7 g, 42% yield). LCMS (ESI) m/z calcd for C₁₄H₂₀BrN₃O₃: 357.07. Found: 358.24/360.22 (M/M+2)⁺.

### Preparation of dimethyl 2-(6-(isobutyl(tetrahydro-2H-pyran-4-yl)amino)-5-nitropyridin-3-yl)malonate

[chemical structure]

A mixture of 5-bromo-N-isobutyl-3-nitro-N-(tetrahydro-2H-pyran-4-yl)pyridin-2-amine (6.0 g, 16.81 mmol), dimethyl malonate (6.66 g, 50.42 mmol), copper iodide (640 mg, 3.36 mmol), picolinic acid (830 mg, 6.80 mmol), Cs₂CO₃ (16.4 g, 50.34 mmol) and dioxane (60 mL) was stirred at 100° C. for 16 h. After cooled to room temperature, the reaction mixture was filtered and the filtrate was partitioned between EtOAc and H₂O. The layers were separated and the organic layer was washed with brine, dried over Na₂SO₄, filtered and concentrated to give the crude product which was purified by flash chromatography (silica gel, 0-30% EtOAc in PE) to afford the title compound (2.8 g, 41% yield). (ESI) m/z calcd for C₁₉H₂₇N₃O₇: 409.18. Found: 410.15 (M+1)⁺.

### Preparation of ethyl 2-(6-(isobutyl(tetrahydro-2H-pyran-4-yl)amino)-5-nitropyridin-3-yl)acetate

[chemical structure]

A mixture of dimethyl 2-(6-(isobutyl(tetrahydro-2H-pyran-4-yl)amino)-5-nitropyridin-3-yl)malonate (2.8 g, 6.85 mmol), KOH (3.84 g, 68.46 mmol) and ethanol (50 mL) was heated at reflux temperature for 2 h. After cooled to room temperature, the reaction mixture was adjusted to pH 4-5 with 6 N HCl. The solvent was removed by evaporation in vacuum, and the resulting residue was extracted with EtOAc. The organic layer was washed with brine and dried over MgSO₄. Filtration and concentration in vacuum gave 2-(6-(isobutyl(tetrahydro-2H-pyran-4-yl)amino)-5-nitropyridin-3-yl)acetic acid as a red solid.

A mixture of above crude acid and concentrated H₂SO₄ (1.5 mL) in EtOH (100 mL) was heated at reflux temperature for 8 h. The solvent was removed by evaporation in vacuum. The residue was diluted with water and extracted with EtOAc. The organics were washed sequentially with sat. aqueous NaHCO₃, water, and brine, and dried over Na₂SO₄. Filtration and concentration in vacuum gave a crude product, which was purified by flash chromatography (silica gel, 0-30% EtOAc in PE) to afford the title compound (2.2 g, 88% yield). (ESI) m/z calcd for C₁₈H₂₇N₃O₅: 365.20. Found: 366.03 (M+1)⁺.

### Preparation of ethyl 2-(6-(isobutyl(tetrahydro-2H-pyran-4-yl)amino)-5-nitropyridin-3-yl)-2-methylpropanoate

[chemical structure]

At 0° C., to a suspension of NaH (247 mg, 6.16 mmol) in DMF (8 mL), a solution of ethyl 2-(6-(isobutyl(tetrahydro-2H-pyran-4-yl)amino)-5-nitropyridin-3-yl)acetate (750 mg, 2.06 mmol) and iodidemethane (729 mg, 5.14 mmol) in ether (2 mL) was added drop wise. The resulting mixture was allowed to warm up to room temperature and stirred overnight. The residue was quenched with saturated aq. NH₄Cl and extracted with EtOAc. The organics were washed sequentially with water and brine, and dried over Na₂SO₄. Filtration and concentration in vacuum gave a crude product, which was purified by flash chromatography (silica gel, 0-30% EtOAc in PE) to afford the title compound (690 mg, 86% yield). (ESI) m/z calcd for C₂₀H₃₁N₃O₅: 393.23. Found: 394.23 (M+1)⁺.

### Preparation of ethyl 2-(5-amino-6-(isobutyl(tetrahydro-2H-pyran-4-yl)amino)pyridin-3-yl)-2-methylpropanoate

[chemical structure]

A mixture of ethyl 2-(6-(isobutyl(tetrahydro-2H-pyran-4-yl)amino)-5-nitropyridin-3-yl)-2-methylpropanoate (690 mg, 1.76 mmol) and 10% Pd/C (700 mg) in EtOAc (10 mL) was stirred at 25° C. under H₂ atmosphere overnight. The resulting mixture was filtered through a pad of Celite and the filtrate was concentrated under reduced pressure to give the crude product, which was purified by flash chromatography (silica gel, 0-40% EtOAc in PE) to afford the title compound (620 mg, 97% yield) as a yellow oil. (ESI) m/z calcd for C₂₀H₃₃N₃O₃: 363.25. Found: 364.02 (M+1)⁺.

### Preparation of ethyl 2-(5-((5-chloropyridin-2-yl)amino)-6-(isobutyl(tetrahydro-2H-pyran-4-yl)amino)pyridin-3-yl)-2-methylpropanoate

[chemical structure]

A mixture of ethyl 2-(5-amino-6-(isobutyl(tetrahydro-2H-pyran-4-yl)amino)pyridin-3-yl)-2-methylpropanoate (620 mg, 1.71 mmol), 2-bromo-5-chloropyridine (657 mg, 3.42 mmol), Pd₂(dba)₃ (312 mg, 0.342 mmol), Xantphos (395 mg, 0.683 mmol) and Cs₂CO₃ (1.11 g, 3.42 mmol) in dioxane (8 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (400 mg, 49% yield). LCMS (ESI) m/z calcd for C₂₅H₃₅ClN₁₄O₃: 474.24. Found: 475.63/477.70 (M/M+2)⁺.

### Example 6

### Preparation of 2-(5-((5-chloropyridin-2-yl)amino)-6-(isobutyl(tetrahydro-2H-pyran-4-yl)amino)pyridin-3-yl)-2-methylpropanoic acid

[chemical structure]

To a solution of ethyl 2-(5-((5-chloropyridin-2-yl)amino)-6-(isobutyl(tetrahydro-2H-pyran-4-yl)amino)pyridin-3-yl)-2-methylpropanoate (60 mg, 0.126 mmol) in MeOH (2 mL) was added 4N NaOH aq. (0.32 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified by HPLC (C18, 60-100% MeCN in H₂O with 0.1% formic acid) to afford the title compound (29 mg, 51% yield) as a white powder. ¹H NMR (400 MHz, DMSO) δ 12.51 (s, 1H), 8.23 (d, J=2.4 Hz, 1H), 8.14 (d, J=2.6 Hz, 1H), 8.06 (s, 1H), 8.01 (d, J=2.4 Hz, 1H), 7.65 (dd, J=8.9, 2.7 Hz, 1H), 6.94 (d, J=8.9 Hz, 1H), 3.83-3.76 (m, 2H), 3.27-3.20 (m, 1H), 3.12-3.03 (m, 2H), 2.95 (d, J=6.8 Hz, 2H), 1.67-1.52 (m, 4H), 1.49 (s, 6H), 1.44-1.37 (m, 1H), 0.79 (d, J=6.6 Hz, 6H). LCMS (ESI) m/z calcd for C₂₃H₃₁ClN₄O₃: 446.21. Found: 447.36/449.67 (M/M+2)⁺.

### Example 7

### Preparation of 2-(5-((5-chloropyridin-2-yl)amino)-6-(isobutyl(tetrahydro-2H-pyran-4-yl)amino)pyridin-3-yl)-2-methyl-N-(methylsulfonyl)propanamide

[chemical structure]

To a solution of 2-(5-((5-chloropyridin-2-yl)amino)-6-(isobutyl(tetrahydro-2H-pyran-4-yl)amino)pyridin-3-yl)-2-methylpropanoic acid (130 mg, 0.29 mmol), methanesulfonamide (33 mg, 0.35 mmol) and DMAP (7 mg, 0.06 mmol) in DCM (3 mL), was added DCC (78 mg, 0.38 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 50-100% MeCN in H₂O with 0.1% formic acid) to afford the title compound (51 mg, 34% yield) as a white powder. ¹H NMR (400 MHz, DMSO) δ 11.43 (s, 1H), 8.13-8.08 (m, J=5.8, 2.5 Hz, 2H), 8.06 (s, 1H), 7.94 (d, J=2.4 Hz, 1H), 7.66 (dd, J=8.9, 2.6 Hz, 1H), 6.97 (d, J=8.9 Hz, 1H), 3.85-3.76 (m, J=10.8 Hz, 2H), 3.28-3.18 (m, 4H), 3.07 (t, J=10.8 Hz, 2H), 2.97 (d, J=6.8 Hz, 2H), 1.70-1.53 (m, 4H), 1.50 (s, 6H), 1.46-1.37 (m, J=13.2, 6.6 Hz, 1H), 0.87-0.75 (m, J=6.6 Hz, 6H). LCMS (ESI) m/z calcd for C₂₄H₃₄ClN₅O₄S: 523.20. Found: 524.25/526.60 (M/M+2)⁺.

### Example 8

### Preparation of 2-(5-((5-chloropyridin-2-yl)amino)-6-(isobutyl(tetrahydro-2H-pyran-4-yl)amino)pyridin-3-yl)-2-methyl-N-((trifluoromethyl)sulfonyl)propanamide

[chemical structure]

To a solution of 2-(5-((5-chloropyridin-2-yl)amino)-6-(isobutyl(tetrahydro-2H-pyran-4-yl)amino)pyridin-3-yl)-2-methylpropanoic acid (130 mg, 0.29 mmol), trifluoromethanesulfonamide (52 mg, 0.35 mmol) and DMAP (7 mg, 0.06 mmol) in DCM (3 mL), was added DCC (78 mg, 0.38 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 40-100% MeCN in H₂O with 0.1% formic acid) to afford the title compound (54 mg, 32% yield) as a white powder. ¹H NMR (400 MHz, DMSO) δ 8.67 (s, 1H), 8.23 (s, 1H), 8.09 (d, J=2.4 Hz, 1H), 7.82 (s, 1H), 7.70 (dd, J=8.9, 2.5 Hz, 1H), 6.94 (d, J=8.9 Hz, 1H), 3.91-3.71 (m, 3H), 3.19-3.02 (m, 4H), 1.78-1.64 (m, 2H), 1.63-1.51 (m, 3H), 1.42 (s, 6H), 0.79 (d, J=6.6 Hz, 6H). The proton of sulfonamide group was not observed. LCMS (ESI) m/z calcd for C₂₄H₃₁ClF₃N₅O₄S: 577.17. Found: 578.25/580.68 (M/M+2)⁺.

[chemical structure]

[chemical structure]

### Preparation of ethyl 1-(6-(isobutyl(tetrahydro-2H-pyran-4-yl)amino)-5-nitropyridin-3-yl)cyclopropane-1-carboxylate

[chemical structure]

To a mixture of ethyl 2-(6-(isobutyl(tetrahydro-2H-pyran-4-yl)amino)-5-nitropyridin-3-yl)acetate (1 g, 2.74 mmol), 1-bromo-2-chloroethane (784 mg, 548 mmol) and benzyltriethylammonium chloride (4.4 g, 19.31 mmol) was added 50% aqueous NaOH (20 mL), and the resulting mixture was heated at 50° C. for 1 h. After cooling, the mixture was poured into water and extracted with EtOAc. The organic layer was washed sequentially with water, 1 N aqueous HCl and brine, and dried over MgSO₄. Filtration, concentration in vacuum afforded the title compound (500 mg, 47%) as a yellow oil, which was used in the following step without further purification. (ESI) m/z calcd for C₂₀H₂₉N₃O₅: 391.21. Found: 392.02 (M+1)⁺.

### Preparation of ethyl 1-(5-amino-6-(isobutyl(tetrahydro-2H-pyran-4-yl)amino)pyridin-3-yl)cyclopropane-1-carboxylate

[chemical structure]

A mixture of ethyl 1-(6-(isobutyl(tetrahydro-2H-pyran-4-yl)amino)-5-nitropyridin-3-yl)cyclopropane-1-carboxylate (500 mg, 1.28 mmol) and 10% Pd/C (500 mg) in EtOAc (10 mL) was stirred at 25° C. under H₂ atmosphere overnight. The resulting mixture was filtered through a pad of Celite and the filtrate was concentrated under reduced pressure to give the crude product which was purified by flash chromatography (silica gel, 0-40% EtOAc in PE) to afford the title compound (420 mg, 91% yield) as a yellow oil. (ESI) m/z calcd for C₂₀H₃₁N₃O₃: 361.24. Found: 362.40 (M+1)⁺.

### Preparation of ethyl 1-(5-((5-chloropyridin-2-yl)amino)-6-(isobutyl(tetrahydro-2H-pyran-4-yl)amino)pyridin-3-yl)cyclopropane-1-carboxylate

[chemical structure]

A mixture of ethyl 1-(5-amino-6-(isobutyl(tetrahydro-2H-pyran-4-yl)amino)pyridin-3-yl)cyclopropane-1-carboxylate (420 mg, 1.16 mmol), 2-bromo-5-chloropyridine (448 mg, 2.33 mmol), Pd₂(dba)₃ (213 mg, 0.233 mmol), Xantphos (269 mg, 0.465 mmol) and Cs₂CO₃ (757 mg, 2.33 mmol) in dioxane (8 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (290 mg, 53% yield). (ESI) m/z calcd for C₂₅H₃₃ClN₄O₃: 472.22. Found: 473.01/475.23 (M/M+2)⁺.

### Example 9

### Preparation of 1-(5-((5-chloropyridin-2-yl)amino)-6-(isobutyl(tetrahydro-2H-pyran-4-yl)amino)pyridin-3-yl)cyclopropane-1-carboxylic acid

[chemical structure]

To a solution of ethyl 1-(5-((5-chloropyridin-2-yl)amino)-6-(isobutyl(tetrahydro-2H-pyran-4-yl)amino)pyridin-3-yl)cyclopropane-1-carboxylate (60 mg, 1.24 mmol) in MeOH (3 mL) was added 4N NaOH aq. (0.32 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified by HPLC (C18, 10-100% MeCN in H₂O with 0.1% formic acid) to afford the title compound (22 mg, 39% yield) as a yellow powder. ¹H NMR (400 MHz, DMSO) δ 12.53 (br, 1H), 8.20 (dd, J=15.4, 2.4 Hz, 2H), 8.03 (s, 1H), 7.94 (d, J=1.7 Hz, 1H), 7.66 (dd, J=8.9, 2.6 Hz, 1H), 6.98 (d, J=8.9 Hz, 1H), 3.85-3.76 (m, 2H), 3.22-3.16 (m, 1H), 3.13-3.05 (m, 2H), 2.95 (d, J=6.8 Hz, 2H), 1.67-1.51 (m, 4H), 1.48-1.37 (m, 3H), 1.19-1.09 (m, 2H), 0.80 (d, J=6.6 Hz, 6H). (ESI) m/z calcd for C₂₃H₂₉ClN₄O₃: 444.19. Found: 445.11/447.29 (M/M+2)⁺.

### Example 10

### Preparation of 1-(5-((5-chloropyridin-2-yl)amino)-6-(isobutyl(tetrahydro-2H-pyran-4-yl)amino)pyridin-3-yl)-N-(methylsulfonyl)cyclopropane-1-carboxamide

[chemical structure]

To a solution of 1-(5-((5-chloropyridin-2-yl)amino)-6-(isobutyl(tetrahydro-2H-pyran-4-yl)amino)pyridin-3-yl)cyclopropane-1-carboxylic acid (150 mg, 0.34 mmol), methanesulfonamide (38 mg, 0.40 mmol) and DMAP (8 mg, 0.07 mmol) in DCM (3 mL), was added DCC (90 mg, 0.44 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 50-100% MeCN in H₂O with 0.1% formic acid) to afford the title compound (32 mg, 18% yield) as a white powder. ¹H NMR (400 MHz, DMSO) δ 11.20 (s, 1H), 8.21-8.15 (m, 2H), 8.03 (s, 1H), 7.92 (d, J=2.2 Hz, 1H), 7.66 (dd, J=8.9, 2.7 Hz, 1H), 7.00 (d, J=8.9 Hz, 1H), 3.84-3.77 (m, 2H), 3.26-3.21 (m, 1H), 3.18 (s, 3H), 3.09 (t, J=10.3 Hz, 2H), 2.96 (d, J=6.8 Hz, 2H), 1.68-1.55 (m, 4H), 1.52-1.46 (m, 2H), 1.45-1.39 (m, 1H), 1.22-1.14 (m, 2H), 0.81 (d, J=6.6 Hz, 6H). (ESI) m/z calcd for C₂₄H₃₂ClN₅O₄S: 521.19. Found: 522.25/524.60 (M/M+2)⁺.

### Example 11

### Preparation of 1-(5-((5-chloropyridin-2-yl)amino)-6-(isobutyl(tetrahydro-2H-pyran-4-yl)amino)pyridin-3-yl)-N-((trifluoromethyl)sulfonyl)cyclopropane-1-carboxamide

[chemical structure]

To a solution of 1-(5-((5-chloropyridin-2-yl)amino)-6-(isobutyl(tetrahydro-2H-pyran-4-yl)amino)pyridin-3-yl)cyclopropane-1-carboxylic acid (150 mg, 0.34 mmol), trifluoromethanesulfonamide (60 mg, 0.40 mmol) and DMAP (8 mg, 0.07 mmol) in DCM (3 mL), was added DCC (90 mg, 0.41 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (68 mg, 35% yield) as a white powder. ¹H NMR (400 MHz, DMSO) δ 8.51 (s, 1H), 8.20 (d, J=1.9 Hz, 1H), 8.14 (d, J=2.5 Hz, 1H), 7.89 (d, J=2.1 Hz, 1H), 7.69 (dd, J=8.9, 2.6 Hz, 1H), 6.97 (d, J=8.9 Hz, 1H), 3.87-3.81 (m, 2H), 3.70-3.54 (m, 1H), 3.16-3.05 (m, 4H), 1.73-1.58 (m, 4H), 1.55-1.46 (m, 1H), 1.41-1.35 (m, 2H), 1.07-1.00 (m, 2H), 0.79 (d, J=6.6 Hz, 6H). The proton of sulfonamide group was not observed. LCMS (ESI) m/z calcd for C₂₄H₂₉ClF₃N₅O₄S: 575.16. Found: 576.25/578.68 (M/M+2)⁺.

[chemical structure]

[chemical structure]

[chemical structure]

### Preparation of 6-bromo-2-nitropyridin-3-amine

[chemical structure]

To a stirred suspension of 2-nitro-pyridin-3-ylamine (25.0 g, 179.7 mmol) and sodium acetate (15.5 g, 188.7 mmol) in acetic acid (150 mL), a solution of bromine (13.8 mL, 269.6 mmol) in acetic acid (50 ml) was added dropwise and the reaction mixture was stirred overnight. The acetic acid was removed under reduced pressure. The residue was cooled to 0° C., neutralized with saturated sodium bicarbonate solution to adjust the pH to ˜7, and extracted with ethyl acetate. The combined organic extracts were washed with brine, dried over anhydrous Na₂SO₄, and concentrated under reduced pressure. The residue was triturated with ethyl acetate to afford compound (34.4 g, 88% yield) as a yellow solid. LCMS (ESI) m/z calcd for 6-bromo-2-nitropyridin-3-amine C₅H₄BrN₃O₂: 216.95. Found: 218.1/220.1 (M/M+2)⁺.

### Preparation of 6-bromo-2-nitro-N-(tetrahydro-2H-pyran-4-yl)pyridin-3-amine

[chemical structure]

At 0° C., to a suspension of 6-bromo-2-nitropyridin-3-amine (34.4 g, 157.8 mmol), tetrahydro-4H-pyran-4-one (39.5 g, 394.5 mmol), acetic acid (170 mL) and THF (340 mL), was added 2 M BH₃ in Me₂S (87 mL, 173.6 mmol) dropwise. After stirred at room temperature for another 2 hours, the mixture was poured into ice-water. The precipitated solid was collected by filtration and dried under reduced pressure at 40° C. overnight to give the title compound (39.2 g, 83% yield) as a yellow solid. LCMS (ESI) m/z calcd for C₁₀H₁₂BrN₃O₃: 301.0. Found: 302.4/304.4 (M/M+2)⁺.

### Preparation of 6-bromo-N-(2-methylallyl)-2-nitro-N-(tetrahydro-2H-pyran-4-yl)pyridin-3-amine

[chemical structure]

At 0° C., to a solution of 6-bromo-2-nitro-N-(tetrahydro-2H-pyran-4-yl)pyridin-3-amine (8.0 g, 26.6 mmol) in DMF (120 mL), was added NaH (2.13 g, 53.2 mmol) portion wise and the resulting mixture was stirred at 0° C. for another 30 min. 3-bromo-2-methylprop-1-ene (7.18 g, 53.2 mmol) was added drop wise and this was stirred at 0° C. for 2 h. The resulting mixture was partitioned between EtOAc and saturated aqueous NH₄Cl. The organic layer was washed with brine, dried over Na₂SO₄, filtered and concentrated to give the crude product which was purified by flash chromatography (silica gel, 0-50% EtOAc in PE) to afford the title compound (5.8 g, 61% yield). LCMS (ESI) m/z calcd for C₁₄H₁₈BrN₃O₃: 355.05. Found: 356.24/358.26 (M/M+2)⁺.

### Preparation of methyl 2-(5-((2-methylallyl)(tetrahydro-2H-pyran-4-yl)amino)-6-nitropyridin-2-yl)acetate

[chemical structure]

A mixture of 6-bromo-N-(2-methylallyl)-2-nitro-N-(tetrahydro-2H-pyran-4-yl)pyridin-3-amine (10.0 g, 28.2 mmol), dimethyl malonate (7.46 g, 56.4 mmol), copper iodide (1.07 g, 5.64 mmol), picolinic acid (694 mg, 5.64 mmol), Cs₂CO₃ (18.4 g, 56.4 mmol) and dioxane (150 mL) was stirred at 100° C. for 16 h. After cooled to room temperature, the reaction mixture was filtered and the filtrate was partitioned between EtOAc and H₂O. The layers were separated and the organic layer was washed with brine, dried over Na₂SO₄, filtered and concentrated to give the crude product which was purified by flash chromatography (silica gel, 0-30% EtOAc in PE) to afford the title compound (4.6 g, 47% yield). (ESI) m/z calcd for C₁₇H₂₃N₃O₅: 349.16. Found: 350.46 (M+1)⁺.

### Preparation of 1-(5-((2-methylallyl)(tetrahydro-2H-pyran-4-yl)amino)-6-nitropyridin-2-yl)cyclopropane-1-carboxylic acid

[chemical structure]

To a mixture of methyl 2-(5-((2-methylallyl)(tetrahydro-2H-pyran-4-yl)amino)-6-nitropyridin-2-yl)acetate (3.0 g, 8.59 mmol), 1-bromo-2-chloroethane (2.46 g, 17.2 mmol), benzyltriethylammonium chloride (13.9 g, 61 mmol) and THF (20 mL) was added 50% aqueous NaOH (20 mL), and the resulting mixture was heated at 50° C. for 1 h. After cooling, the mixture was poured into ice-water and neutralized with 6 N HCl. The resulting mixture was extracted with EtOAc. The organic layer was separated, washed sequentially with water, 1 N aqueous HCl and brine, and dried over MgSO₄. Filtration, concentration in vacuum afforded the title compound (2.4 g, 77%), which was used in the following step without further purification. (ESI) m/z calcd for C₁₈H₂₃N₃O₅: 361.16. Found: 362.43 (M+1)⁺.

### Preparation of methyl 1-(5-((2-methylallyl)(tetrahydro-2H-pyran-4-yl)amino)-6-nitropyridin-2-yl)cyclopropane-1-carboxylate

[chemical structure]

At 0° C., a solution of 1-(5-((2-methylallyl)(tetrahydro-2H-pyran-4-yl)amino)-6-nitropyridin-2-yl)cyclopropane-1-carboxylic acid (2.4 g, 6.65 mmol) in MeOH (24 mL) was added SOCl₂ (1.5 mL, 19.95 mmol) dropwise. The resulting mixture was stirred at room temperature for 18 h. The solvent was removed by evaporation in vacuum. The residue was diluted with water and extracted with EtOAc. The organics were washed sequentially with sat. aqueous NaHCO₃, water and brine, and dried over Na₂SO₄. Filtration and concentration in vacuum gave a crude product, which was purified by flash chromatography (silica gel, 0-50% EtOAc in PE) to afford the title compound (2.0 g, 80% yield). (ESI) m/z calcd for C₁₉H₂₅N₃O₅: 375.18. Found: 376.20 (M+1)⁺.

### Preparation of methyl 1-(5-(isobutyl(tetrahydro-2H-pyran-4-yl)amino)-6-nitropyridin-2-yl)cyclopropane-1-carboxylate

[chemical structure]

A mixture of methyl 1-(5-((2-methylallyl)(tetrahydro-2H-pyran-4-yl)amino)-6-nitropyridin-2-yl)cyclopropane-1-carboxylate (2.0 g, 5.3 mmol), 4-methylbenzenesulfonohydrazide (7.9 g, 42.4 mmol) and xylene (20 mL) was stirred at 110° C. for 16 h. After cooled to room temperature, the reaction mixture was filtered and the filtrate was partitioned between EtOAc and H₂O. The layers were separated and the organic layer was washed with brine, dried over Na₂SO₄, filtered and concentrated to give the crude product which was purified by flash chromatography (silica gel, 0-40% EtOAc in PE) to afford the title compound (1.0 g, 50% yield). (ESI) m/z calcd for C₁₉H₂₇N₃O₅: 377.20. Found: 378.44 (M+1)⁺.

### Preparation of methyl 1-(6-amino-5-(isobutyl(tetrahydro-2H-pyran-4-yl)amino)pyridin-2-yl)cyclopropane-1-carboxylate

[chemical structure]

A mixture of methyl 1-(5-(isobutyl(tetrahydro-2H-pyran-4-yl)amino)-6-nitropyridin-2-yl)cyclopropane-1-carboxylate (200 mg, 0.52 mmol), SnCl₂ (1.08 g, 5.2 mmol), Et₃N (3.0 mL, 15.6 mmol) and EtOH (6 mL) was stirred at 80° C. for 3 h. The resulting mixture was filtered through a pad of Celite and the filtrate was concentrated under reduced pressure to give the crude product, which was purified by flash chromatography (silica gel, 0-50% EtOAc in PE) to afford the title compound (128 mg, 71% yield). (ESI) m/z calcd for C₁₉H₂₉N₃O₃: 347.22. Found: 348.45 (M+1)⁺.

### Preparation of methyl 1-(6-((5-chloropyridin-2-yl)amino)-5-(isobutyl(tetrahydro-2H-pyran-4-yl)amino)pyridin-2-yl)cyclopropane-1-carboxylate

[chemical structure]

A mixture of methyl 1-(6-amino-5-(isobutyl(tetrahydro-2H-pyran-4-yl)amino)pyridin-2-yl)cyclopropane-1-carboxylate (130 mg, 0.38 mmol), 2-bromo-5-chloropyridine (147 mg, 0.76 mmol), Pd₂(dba)₃ (35 mg, 0.038 mmol), Xantphos (44 mg, 0.076 mmol) and Cs₂CO₃ (248 mg, 0.76 mmol) in dioxane (3 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (106 mg, 61% yield). LCMS (ESI) m/z calcd for C₂₄H₃₁ClN₄O₃: 458.21. Found: 460.48/461.34 (M/M+2)⁺.

### Example 15

### Preparation of 1-(6-((5-chloropyridin-2-yl)amino)-5-(isobutyl(tetrahydro-2H-pyran-4-yl)amino)pyridin-2-yl)cyclopropane-1-carboxylic acid

[chemical structure]

To a solution of methyl 1-(6-((5-chloropyridin-2-yl)amino)-5-(isobutyl(tetrahydro-2H-pyran-4-yl)amino)pyridin-2-yl)cyclopropane-1-carboxylate (103 mg, 0.224 mmol) in MeOH (1.0 mL) was added 4N NaOH aq. (1.0 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified by Prep. HPLC (C18, 30-100% MeCN in H₂O with 0.1% formic acid) to afford the title compound (90 mg, 90% yield). ¹H NMR (400 MHz, DMSO) δ 12.61 (s, 1H), 8.67 (s, 1H), 8.38 (d, J=9.0 Hz, 1H), 8.26 (d, J=2.5 Hz, 1H), 7.91-7.84 (m, 1H), 7.62 (d, J=8.0 Hz, 1H), 7.13 (d, J=8.0 Hz, 1H), 3.88-3.78 (m, 2H), 3.25-3.17 (m, 2H), 2.92-2.77 (m, 3H), 1.75-1.64 (m, 2H), 1.56-1.44 (m, 4H), 1.41-1.28 (m, 3H), 0.84 (d, J=6.5 Hz, 6H). LCMS (ESI) m/z calcd for C₂₃H₂₉ClN₄O₃: 444.19. Found: 445.33/447.30 (M/M+2)⁺.

### Example 16

### Preparation of 1-(6-((5-chloropyridin-2-yl)amino)-5-(isobutyl(tetrahydro-2H-pyran-4-yl)amino)pyridin-2-yl)-N-(methylsulfonyl)cyclopropane-1-carboxamide

[chemical structure]

To a solution of 1-(6-((5-chloropyridin-2-yl)amino)-5-(isobutyl(tetrahydro-2H-pyran-4-yl)amino)pyridin-2-yl)cyclopropane-1-carboxylic acid (50 mg, 0.11 mmol), methanesulfonamide (12 mg, 0.12 mmol) and DMAP (3 mg, 0.022 mmol) in THF (1 mL), was added DCC (27 mg, 0.132 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 20-80% MeCN in H₂O with 0.1% formic acid) to afford the title compound (21 mg, 36% yield) as a white solid. ¹H NMR (400 MHz, DMSO) δ 11.88 (s, 1H), 8.70 (s, 1H), 8.39 (d, J=9.0 Hz, 1H), 8.26 (d, J=2.4 Hz, 1H), 7.86-7.78 (m, 1H), 7.63 (d, J=8.1 Hz, 1H), 6.80 (d, J=7.8 Hz, 1H), 3.90-3.78 (m, 2H), 3.25-3.18 (m, 5H), 2.93-2.76 (m, 3H), 1.74-1.63 (m, 2H), 1.60-1.43 (m, 4H), 1.40-1.29 (m, 3H), 0.85 (d, J=6.4 Hz, 6H). LCMS (ESI) m/z calcd for C₂₄H₃₂ClN₅O₄S: 521.19. Found: 522.66/524.64 (M/M+2)⁺.

[chemical structure]

[chemical structure]

### Preparation of methyl 2-methyl-2-(5-((2-methylallyl)(tetrahydro-2H-pyran-4-yl)amino)-6-nitropyridin-2-yl)propanoate

[chemical structure]

At 0° C., to a suspension of NaH (510 mg, 12.9 mmol) in DMF (20 mL), a solution of methyl 2-(5-((2-methylallyl)(tetrahydro-2H-pyran-4-yl)amino)-6-nitropyridin-2-yl)acetate (1.5 g, 4.3 mmol) and iodidemethane (1.8 g, 12.9 mmol) in ether (5 mL) was added drop wise. The resulting mixture was allowed to warm up to room temperature and stirred overnight. The residue was quenched with saturated aq. NH₄Cl and extracted with EtOAc. The organics were washed sequentially with water and brine, and dried over Na₂SO₄. Filtration and concentration in vacuum gave a crude product, which was purified by flash chromatography (silica gel, 0-30% EtOAc in PE) to afford the title compound (1.6 g, 96% yield). (ESI) m/z calcd for C₁₉H₂₇N₃O₅: 377.20. Found: 378.22 (M+1)⁺.

### Preparation of methyl 2-(6-amino-5-(isobutyl(tetrahydro-2H-pyran-4-yl)amino)pyridin-2-yl)-2-methylpropanoate

[chemical structure]

A mixture of methyl 2-methyl-2-(5-((2-methylallyl)(tetrahydro-2H-pyran-4-yl)amino)-6-nitropyridin-2-yl)propanoate (1.6 g, 4.4 mmol) and 10% Pd/C (500 mg) in EtOAc (20 mL) was stirred at 25° C. under H₂ atmosphere overnight. The resulting mixture was filtered through a pad of Celite and the filtrate was concentrated under reduced pressure to give the crude product, which was purified by flash chromatography (silica gel, 0-40% EtOAc in PE) to afford the title compound (560 mg, 36% yield) as a yellow oil. (ESI) m/z calcd for C₁₉H₃₁N₃O₃: 349.24. Found: 350.79 (M+1)⁺.

### Preparation of methyl 2-(6-((5-chloropyridin-2-yl)amino)-5-(isobutyl(tetrahydro-2H-pyran-4-yl)amino)pyridin-2-yl)-2-methylpropanoate

[chemical structure]

A mixture of methyl 2-(6-amino-5-(isobutyl(tetrahydro-2H-pyran-4-yl)amino)pyridin-2-yl)-2-methylpropanoate (560 mg, 1.60 mmol), 2-bromo-5-chloropyridine (544 mg, 3.2 mmol), Pd₂(dba)₃ (140 mg, 0.16 mmol), Xantphos (196 mg, 0.32 mmol) and Cs₂CO₃ (1.11 g, 0.16 mmol) in dioxane (6 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (400 mg, 49% yield). LCMS (ESI) m/z calcd for C₂₄H₃₃ClN₄O₃: 460.22. Found: 461.12/463.14 (M/M+2)⁺.

### Example 17

### Preparation of 2-(6-((5-chloropyridin-2-yl)amino)-5-(isobutyl(tetrahydro-2H-pyran-4-yl)amino)pyridin-2-yl)-2-methylpropanoic acid

[chemical structure]

To a solution of methyl 2-(6-((5-chloropyridin-2-yl)amino)-5-(isobutyl(tetrahydro-2H-pyran-4-yl)amino)pyridin-2-yl)-2-methylpropanoate (560 mg, 1.21 mmol) in MeOH (4.0 mL) was added 4N NaOH aq. (2.0 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified by HPLC (C18, 60-100% MeCN in H₂O with 0.1% formic acid) to afford the title compound (510 mg, 94% yield) as a white powder. ¹H NMR (400 MHz, DMSO) δ 12.34 (s, 1H), 8.67 (s, 1H), 8.52 (d, J=9.0 Hz, 1H), 8.26 (d, J=2.2 Hz, 1H), 7.82 (dd, J=9.0, 2.5 Hz, 1H), 7.66 (d, J=8.0 Hz, 1H), 6.94 (d, J=8.0 Hz, 1H), 3.88-3.78 (m, 2H), 3.22 (t, J=11.5 Hz, 2H), 2.95-2.75 (m, 3H), 1.74-1.63 (m, 2H), 1.63-1.42 (m, 8H), 1.36-1.28 (m, 1H), 0.85 (d, J=6.3 Hz, 6H). LCMS (ESI) m/z calcd for C₂₃H₃₁ClN₄O₃: 446.21. Found: 447.18/449.23 (M/M+2)⁺.

### Example 18

### Preparation of 2-(6-((5-chloropyridin-2-yl)amino)-5-(isobutyl(tetrahydro-2H-pyran-4-yl)amino)pyridin-2-yl)-2-methyl-N-(methylsulfonyl)propanamide

[chemical structure]

To a solution of 2-(6-((5-chloropyridin-2-yl)amino)-5-(isobutyl(tetrahydro-2H-pyran-4-yl)amino)pyridin-2-yl)-2-methylpropanoic acid (130 mg, 0.29 mmol), methanesulfonamide (33 mg, 0.35 mmol) and DMAP (7 mg, 0.06 mmol) in DCM (3 mL), was added DCC (78 mg, 0.38 mmol) in one portion. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 50-100% MeCN in H₂O with 0.1% formic acid) to afford the title compound (51 mg, 34% yield) as a white powder. ¹H NMR (400 MHz, DMSO) δ 11.33 (s, 1H), 8.69 (s, 1H), 8.45 (d, J=9.0 Hz, 1H), 8.25 (d, J=2.4 Hz, 1H), 7.77 (dd, J=9.0, 2.5 Hz, 1H), 7.70 (d, J=8.1 Hz, 1H), 6.91 (d, J=8.0 Hz, 1H), 3.90-3.79 (m, 2H), 3.29-3.10 (m, 5H), 2.98-2.78 (m, 3H), 1.76-1.65 (m, 2H), 1.61-1.47 (m, 8H), 1.40-1.32 (m, 1H), 0.85 (d, J=6.4 Hz, 6H). LCMS (ESI) m/z calcd for C₂₄H₃₄ClN₅O₄S: 523.20. Found: 524.49/526.47 (M/M+2)⁺.

### Example 20

[chemical structure]

[chemical structure]

### Preparation of methyl 1-(4-(isobutyl(tetrahydro-2H-pyran-4-yl)amino)-3-((2-methoxypyrimidin-5-yl)amino)phenyl)cyclopropane-1-carboxylate

[chemical structure]

A mixture of methyl 1-(3-amino-4-(isobutyl(tetrahydro-2H-pyran-4-yl)amino)phenyl) cyclopropane-1-carboxylate (550 mg, 1.59 mmol), 5-bromo-2-methoxypyrimidine (385 mg, 2.06 mmol), Pd₂(dba)₃ (143 mg, 0.159 mmol), Xantphos (187 mg, 0.318 mmol) and Cs₂CO₃ (1.55 g, 4.76 mmol) in dioxane (10 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (450 mg, 62% yield). LCMS (ESI) m/z calcd for C₂₅H₃₄N₄O₄: 454.26. Found: 455.37 (M+1)⁺.

### Preparation of 1-(4-(isobutyl(tetrahydro-2H-pyran-4-yl)amino)-3-((2-methoxy pyrimidin-5-yl)amino)phenyl)cyclopropane-1-carboxylic acid

[chemical structure]

To a solution of methyl 1-(4-(isobutyl(tetrahydro-2H-pyran-4-yl)amino)-3-((2-methoxypyrimidin-5-yl)amino)phenyl)cyclopropane-1-carboxylate (450 mg, 0.99 mmol) in MeOH (4 mL) was added 4N NaOH aq. (1 mL). After stirred at 25° C. overnight, the resulting mixture was neutralized with 1N HCl and extracted with EtOAc. The organic layer was washed with brine, dried over Na₂SO₄, filtered and concentrated to give the the title compound (436 mg, 100% yield) as a pale solid, which was used in the following step without purification. LCMS (ESI) m/z calcd for C₂₄H₃₂N₄O₄: 440.24. Found: 441.35 (M+1)⁺.

### Preparation of 1-(4-(isobutyl(tetrahydro-2H-pyran-4-yl)amino)-3-((2-methoxy pyrimidin-5-yl)amino)phenyl)-N-(methylsulfonyl)cyclopropane-1-carboxamide

[chemical structure]

To a solution of 1-(4-(isobutyl(tetrahydro-2H-pyran-4-yl)amino)-3-((2-methoxy pyrimidin-5-yl)amino)phenyl)cyclopropane-1-carboxylic acid (200 mg, 0.454 mmol) in THF (2 mL), was added CDI (110 mg, 0.545 mmol) and the resulting mixture was heated at 50° C. After 2 hours, the mixture was cooled down to room temperature, methanesulfonamide (50 mg, 0.49 mmol) and DBU (0.15 mL, 0.908 mmol) in THF (1 mL) was added. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (112 mg, 48% yield) as a white powder. ¹H NMR (400 MHz, DMSO) δ 10.99 (s, 1H), 8.50 (s, 2H), 7.16 (d, J=8.2 Hz, 1H), 7.09 (s, 1H), 6.92 (d, J=2.0 Hz, 1H), 6.76 (dd, J=8.1, 2.0 Hz, 1H), 3.89 (s, 3H), 3.83 (dd, J=11.1, 3.6 Hz, 2H), 3.22-3.11 (m, 5H), 2.92-2.84 (m, 1H), 2.79 (d, J=6.6 Hz, 2H), 1.76-1.68 (m, 2H), 1.60-1.49 (m, 2H), 1.42-1.34 (m, 3H), 1.13-1.05 (m, 2H), 0.83 (d, J=6.6 Hz, 6H). LCMS (ESI) m/z calcd for C₂₅H₃₅N₅O₅S: 517.24. Found: 518.74 (M+1)⁺.

### Example 21

[chemical structure]

[chemical structure]

### Preparation of methyl 1-(4-(isobutyl(tetrahydro-2H-pyran-4-yl)amino)-3-((6-(methoxymethyl)pyridin-3-yl)amino)phenyl)cyclopropane-1-carboxylate

[chemical structure]

A mixture of methyl 1-(3-amino-4-(isobutyl(tetrahydro-2H-pyran-4-yl)amino)phenyl) cyclopropane-1-carboxylate (500 mg, 1.44 mmol), 5-bromo-2-methoxypyrimidine (437 mg, 2.16 mmol), Pd₂(dba)₃ (138 mg, 0.15 mmol), Xantphos (168 mg, 0.29 mmol) and Cs₂CO₃ (939 mg, 2.88 mmol) in dioxane (5 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (435 mg, 65% yield). LCMS (ESI) m/z calcd for C₂₇H₃₇N₃O₄: 467.28. Found: 468.37 (M+1)⁺.

### Preparation of 1-(4-(isobutyl(tetrahydro-2H-pyran-4-yl)amino)-3-((6-(methoxymethyl)pyridin-3-yl)amino)phenyl)cyclopropane-1-carboxylic acid

[chemical structure]

To a solution of methyl 1-(4-(isobutyl(tetrahydro-2H-pyran-4-yl)amino)-3-((6-(methoxymethyl)pyridin-3-yl)amino)phenyl)cyclopropane-1-carboxylate (435 mg, 0.93 mmol) in MeOH (4 mL) was added 4N NaOH aq. (1 mL). After stirred at 25° C. for 4 h, the resulting mixture was neutralized with 1N HCl and extracted with EtOAc. The organic layer was washed with brine, dried over Na₂SO₄, filtered and concentrated to give the title compound (378 mg, 90% yield) as a pale solid, which was used in the following step without purification. LCMS (ESI) m/z calcd for C₂₆H₃₅N₃O₄: 453.26. Found: 454.38 (M+1)⁺.

### Preparation of 1-(4-(isobutyl(tetrahydro-2H-pyran-4-yl)amino)-3-((6-(methoxymethyl)pyridin-3-yl)amino)phenyl)-N-(methylsulfonyl)cyclopropane-1-carboxamide

[chemical structure]

To a solution of 1-(4-(isobutyl(tetrahydro-2H-pyran-4-yl)amino)-3-((6-(methoxymethyl)pyridin-3-yl)amino)phenyl)cyclopropane-1-carboxylic acid (180 mg, 0.4 mmol) in THF (2 mL), was added CDI (130 mg, 0.8 mmol) and the resulting mixture was heated at 50° C. After 2 hours, the mixture was cooled down to room temperature, methanesulfonamide (76 mg, 0.8 mmol) and DBU (122 mg, 0.8 mmol) in THF (1 mL) was added. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (114 mg, 54% yield) as a white powder. ¹H NMR (400 MHz, DMSO) δ 11.10 (s, 1H), 8.39 (d, J=2.6 Hz, 1H), 7.74 (dd, J=8.6, 2.3 Hz, 1H), 7.59 (s, 1H), 7.46 (d, J=8.6 Hz, 1H), 7.23-7.16 (m, 2H), 6.91 (dd, J=8.2, 1.9 Hz, 1H), 4.49 (s, 2H), 3.81 (dd, J=11.0, 3.2 Hz, 2H), 3.35 (s, 3H), 3.22 (s, 3H), 3.09 (t, J=11.0 Hz, 2H), 2.94-2.86 (m, 1H), 2.79 (d, J=6.6 Hz, 2H), 1.67-1.52 (m, 4H), 1.45-1.35 (m, 3H), 1.18-1.13 (m, 2H), 0.81 (d, J=6.6 Hz, 6H). LCMS (ESI) m/z calcd for C₂₇H₃₈N₄O₅S: 530.26. Found: 531.33 (M+1)⁺.

### Example 22 and Example 23

[chemical structure]

[chemical structure]

### Preparation of methyl 1-(3-((2-cyanopyrimidin-5-yl)amino)-4-(isobutyl(tetrahydro-2H-pyran-4-yl)amino)phenyl)cyclopropane-1-carboxylate

[chemical structure]

A mixture of methyl 1-(3-amino-4-(isobutyl(tetrahydro-2H-pyran-4-yl)amino)phenyl) cyclopropane-1-carboxylate (600 mg, 1.73 mmol), 5-bromo-2-methoxypyrimidine (478 mg, 2.60 mmol), Pd₂(dba)₃ (158 mg, 0.17 mmol), Xantphos (200 mg, 0.35 mmol) and K2CO₃ (717 mg, 5.20 mmol) in toluene (10 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (720 mg, 93% yield). LCMS (ESI) m/z calcd for C₂₅H₃₁N₅O₃: 449.24. Found: 450.38 (M+1)⁺.

### Preparation of 1-(3-((2-cyanopyrimidin-5-yl)amino)-4-(isobutyl(tetrahydro-2H-pyran-4-yl)amino)phenyl)cyclopropane-1-carboxylic acid

[chemical structure]

To a solution of methyl 1-(3-((2-cyanopyrimidin-5-yl)amino)-4-(isobutyl(tetrahydro-2H-pyran-4-yl)amino)phenyl)cyclopropane-1-carboxylate (720 mg, 1.60 mmol) in THF (7 mL) was added 1N LiOH aq. (6.4 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified to give the title compound (270 mg, 39% yield) as a pale solid. LCMS (ESI) m/z calcd for C₂₄H₂₉N₅O₃: 435.23. Found: 436.35 (M+1)⁺.

### Preparation of 1-(3-((2-cyanopyrimidin-5-yl)amino)-4-(isobutyl(tetrahydro-2H-pyran-4-yl)amino)phenyl)-N-(methylsulfonyl)cyclopropane-1-carboxamide

[chemical structure]

To a solution of 1-(3-((2-cyanopyrimidin-5-yl)amino)-4-(isobutyl(tetrahydro-2H-pyran-4-yl)amino)phenyl)cyclopropane-1-carboxylic acid (110 mg, 0.253 mmol) in THF (2 mL), was added CDI (82 mg, 0.505 mmol) and the resulting mixture was heated at 50° C. After 2 hours, the mixture was cooled down to room temperature, methanesulfonamide (60 mg, 0.631 mmol) and DBU (77 mg, 0.505 mmol) in THF (1 mL) was added. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (68 mg, 52% yield) as a white powder. ¹H NMR (400 MHz, DMSO) δ 11.08 (s, 1H), 8.53 (s, 2H), 8.28 (s, 1H), 7.28 (d, J=2.1 Hz, 1H), 7.21 (d, J=8.4 Hz, 1H), 7.09 (dd, J=8.3, 2.1 Hz, 1H), 3.85-3.76 (m, 2H), 3.22 (s, 3H), 3.11-3.02 (m, 2H), 3.01-2.94 (m, 1H), 2.77 (d, J=6.6 Hz, 2H), 1.61-1.48 (m, 4H), 1.46-1.35 (m, 3H), 1.20-1.13 (m, 2H), 0.78 (d, J=6.6 Hz, 6H). LCMS (ESI) m/z calcd for C₂₅H₃₂N₆O₄S: 512.22. Found: 513.45 (M+1)⁺.

### Preparation of 5-((2-(isobutyl(tetrahydro-2H-pyran-4-yl)amino)-5-(1-((methylsulfonyl)carbamoyl)cyclopropyl)phenyl)amino)pyrimidine-2-carboxamide

[chemical structure]

At 0° C., to a suspension of 1-(3-((2-cyanopyrimidin-5-yl)amino)-4-(isobutyl(tetrahydro-2H-pyran-4-yl)amino)phenyl)-N-methylsulfonyl)cyclopropane-1-carboxamide (150 mg, 0.29 mmol) and K2CO3 (121 mg, 0.878 mmol) in DMSO (2 mL), was added H₂O₂ (0.5 mL). After stirred at room temperature for 30 min, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (101 mg, 65% yield) as a yellow powder. ¹H NMR (400 MHz, DMSO) δ 11.08 (s, 1H), 8.64 (s, 2H), 7.92 (s, 1H), 7.76 (s, 1H), 7.49 (s, 1H), 7.29 (d, J=2.0 Hz, 1H), 7.22 (d, J=8.4 Hz, 1H), 7.01 (dd, J=8.3, 2.0 Hz, 1H), 3.81 (d, J=10.9 Hz, 2H), 3.22 (s, 3H), 3.08 (t, J=10.6 Hz, 2H), 2.97-2.91 (m, 1H), 2.79 (d, J=6.7 Hz, 2H), 1.65-1.50 (m, 4H), 1.46-1.36 (m, 3H), 1.19-1.12 (m, 2H), 0.81 (d, J=6.6 Hz, 6H). LCMS (ESI) m/z calcd for C₂₅H₃₄N₆O₅S: 530.23. Found: 531.29 (M+1)+.

### Example 24 and Example 25

[chemical structure]

[chemical structure]

[chemical structure]

[chemical structure]

### Preparation of 2-bromo-5-fluoropyridine 1-oxide

[chemical structure]

2-Bromo-5-fluoropyridine (5 g, 28.4 mmol), trifluoroacetic acid (23 mL) and hydrogen peroxide (35% in water) (3 mL, 34.1 mmol) were stirred overnight at 70° C. The mixture was poured into water and extracted with dichloromethane. The organic layers were washed with NaHCO₃(aq), dried over MgSO4 and the solvent was removed under reduced pressure to give the title compound (6 g, 100% yield), which was used in the following step without purification. LCMS (ESI) m/z calcd for C₅H₃BrFNO: 190.94. Found: 192.45/194.44 (M/M+2)⁺.

### Preparation of 2-bromo-5-fluoro-4-nitropyridine 1-oxide

[chemical structure]

At 0° C., fuming nitric acid (2.0 mL) was added to a mixture of 2-bromo-5-fluoropyridine 1-oxide (6 g, 31.3 mmol) and conc. sulfuric acid (30 mL). After stirred at 0° C. for 30 min, the mixture was heated to 100° C. and stirred at this temperature for 4 hours. The reaction mixture was poured into water at 0° C. and adjusted to pH 2 by adding conc. ammonia. The precipitated solid was collected by filtration, washed with water and dried overnight at ambient temperature to afford the title compound (2.5 g, 34% yield). LCMS (ESI) m/z calcd for C₅H₂BrFN₂O₃: 235.92. Found: 237.01/238.99 (M/M+2)⁺.

### Preparation of 2-bromo-5-(isobutyl(tetrahydro-2H-pyran-4-yl)amino)-4-nitropyridine 1-oxide

[chemical structure]

A mixture of 2-bromo-5-fluoro-4-nitropyridine 1-oxide (2 g, 16.9 mmol), N-isobutyltetrahydro-2H-pyran-4-amine (1.6 g, 20.3 mmol) and NMP was stirred at 60° C. under N₂ atmosphere for 18 hr. The resulting mixture was partitioned between EtOAc and H₂O. The layers were separated and the organic layer was washed with brine, dried over Na₂SO₄, filtered and concentrated to give the crude product which was purified by flash chromatography (silica gel, 0-10% EtOAc in PE) to afford the title compound (3 g, 77% yield). LCMS (ESI) m/z calcd for C₁₄H₂₀ BrN₃O₄: 373.06. Found: 374.32/376.30 (M/M+2)⁺.

### Preparation of 6-bromo-N-isobutyl-4-nitro-N-(tetrahydro-2H-pyran-4-yl)pyridin-3-amine

[chemical structure]

A mixture of 2-bromo-5-(isobutyl(tetrahydro-2H-pyran-4-yl)amino)-4-nitropyridine 1-oxide (3 g, 8.0 mmol), Bis(pinacolato)diboron (8 g, 32.1 mmol) and dioxane was stirred at 100° C. under N₂ atmosphere for 18 hr. The resulting mixture was partitioned between EtOAc and H₂O. The layers were separated and the organic layer was washed with brine, dried over Na₂SO₄, filtered and concentrated to give the crude product which was purified by flash chromatography (silica gel, 0-10% EtOAc in PE) to afford the title compound (1.7 g, 59% yield). LCMS (ESI) m/z calcd for C₁₄H₂₀BrN₃O₃: 357.07. Found: 358.12/360.34 (M/M+2)⁺.

### Preparation of methyl 2-(5-(isobutyl(tetrahydro-2H-pyran-4-yl)amino)-4-nitropyridin-2-yl)acetate

[chemical structure]

A mixture of 6-bromo-N-isobutyl-4-nitro-N-(tetrahydro-2H-pyran-4-yl)pyridin-3-amine (23 g, 64.2 mmol), dimethyl malonate (25.3 g, 191.5 mmol), copper iodide (11.5 g, 60.4 mmol), NaI (20 g, 107.6 mmol), N¹,N²-dimethylethane-1,2-diamine (7 g, 79.4 mmol), Cs₂CO₃ (62 g, 190.3 mmol) and dioxane (400 mL) was stirred at 100° C. for 16 h. After cooled to room temperature, the reaction mixture was filtered and the filtrate was partitioned between EtOAc and H₂O. The layers were separated and the organic layer was washed with brine, dried over Na₂SO₄, filtered and concentrated to give the crude product which was purified by flash chromatography (silica gel, 0-30% EtOAc in PE) to afford the title compound (4 g, 18% yield). (ESI) m/z calcd for C₁₇H₂₅N₃O₅: 351.18. Found: 352.27 (M+1)⁺.

### Preparation of 1-(5-(isobutyl(tetrahydro-2H-pyran-4-yl)amino)-4-nitropyridin-2-yl)cyclopropane-1-carboxylic acid

[chemical structure]

To a mixture of ethyl 2-(6-(isobutyl(tetrahydro-2H-pyran-4-yl)amino)-5-nitropyridin-3-yl)acetate (1.5 g, 4.27 mmol), 1-bromo-2-chloroethane (1.2 g, 8.39 mmol) and benzyltriethylammonium chloride (6.9 g, 30.3 mmol) was added 50% aqueous NaOH (20 mL), and the resulting mixture at room temperature for 1 h. After cooling, the mixture was poured into water and extracted with diisopropyl ether. The organic layer was washed sequentially with water, 1 N aqueous HCl and brine, and dried over MgSO4. Filtration, concentration in vacuo afforded the title compound (730 mg, 47%) as a yellow oil, which was used in the following step without further purification. (ESI) m/z calcd for C₁₈H₂₅N₃O₅: 363.18. Found: 364.31 (M+1)⁺.

### Preparation of methyl 1-(5-(isobutyl(tetrahydro-2H-pyran-4-yl)amino)-4-nitropyridin-2-yl)cyclopropane-1-carboxylate

[chemical structure]

At 0° C., to a mixture of 1-(5-(isobutyl(tetrahydro-2H-pyran-4-yl)amino)-4-nitropyridin-2-yl)cyclopropane-1-carboxylic acid (730 mg, 2.01 mmol) in MeOH (10 mL) was added SOCl₂ (1 mL) drop wise and then the resulting mixture was stirred at room temperature overnight. The mixture was poured into water and extracted with EtOAc. The organic layer was washed brine, dried over MgSO₄, concentrated in vacuum to afford a residue, which was purified by chromatography on silica gel to give the title compound (400 mg, 53%) as a yellow oil. (ESI) m/z calcd for C₁₉H₂₇N₃O₅: 377.20. Found: 378.34 (M+1)⁺.

### Preparation of methyl 1-(4-amino-5-(isobutyl(tetrahydro-2H-pyran-4-yl)amino) pyridin-2-yl)cyclopropane-1-carboxylate

[chemical structure]

A suspension of ethyl 1-(6-(isobutyl(tetrahydro-2H-pyran-4-yl)amino)-5-nitropyridin-3-yl)cyclopropane-1-carboxylate (400 mg, 1.06 mmol), zinc powder (347 mg, 5.30 mmol) and NH₄Cl (284 mg, 5.30 mmol) in MeOH (5 mL) was stirred at 65° C. under nitrogen atmosphere overnight. The resulting mixture was filtered through a pad of Celite and the filtrate was concentrated under reduced pressure to give the crude product which was purified by flash chromatography (silica gel, 0-40% EtOAc in PE) to afford the title compound (210 mg, 57% yield) as a yellow oil. (ESI) m/z calcd for C₁₉H₂₉N₃O₃: 347.22. Found: 348.43 (M+1)⁺.

### Preparation of methyl 1-(4-((5-chloropyridin-2-yl)amino)-5-(isobutyl(tetrahydro-2H-pyran-4-yl)amino)pyridin-2-yl)cyclopropane-1-carboxylate

[chemical structure]

A mixture of ethyl 1-(5-amino-6-(isobutyl(tetrahydro-2H-pyran-4-yl)amino)pyridin-3-yl)cyclopropane-1-carboxylate (170 mg, 0.49 mmol), 2-bromo-5-chloropyridine (153 mg, 0.80 mmol), Pd₂(dba)₃ (51 mg, 0.056 mmol), Xantphos (64 mg, 0.11 mmol) and Cs₂CO₃ (460 mg, 1.41 mmol) in dioxane (4 mL) was stirred at 100° C. under N₂ atmosphere overnight. The resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (110 mg, 49% yield). (ESI) m/z calcd for C₂₄H₃₁ClN₄O₃: 458.21. Found: 459.34/461.33(M/M+2)⁺.

### Preparation of 1-(4-((5-chloropyridin-2-yl)amino)-5-(isobutyl(tetrahydro-2H-pyran-4-yl)amino)pyridin-2-yl)cyclopropane-1-carboxylic acid

[chemical structure]

To a solution of methyl 1-(4-((5-chloropyridin-2-yl)amino)-5-(isobutyl(tetrahydro-2H-pyran-4-yl)amino)pyridin-2-yl)cyclopropane-1-carboxylate (40 mg, 0.087 mmol) in MeOH (1 mL) was added 4N NaOH aq. (1 mL). After stirred at 25° C. overnight, the resulting mixture was neutralized with 1N HCl and extracted with EtOAc. The organic layer was washed with brine, dried over Na₂SO₄, filtered and concentrated to give the crude product, which was purified by HPLC (C18, 10-100% MeCN in H₂O with 0.1% formic acid) to afford the title compound (18 mg, 46% yield) as a white solid. ¹H NMR (400 MHz, CDCl₃) δ 8.48 (s, 1H), 8.32 (d, J=2.4 Hz, 1H), 8.14 (s, 1H), 8.10 (s, 1H), 7.59 (dd, J=8.7, 2.6 Hz, 1H), 6.70 (d, J=8.4 Hz, 1H), 4.05-3.93 (m, 2H), 3.39-3.26 (m, 2H), 3.01-2.79 (m, 3H), 2.12-2.03 (m, 2H), 1.74-1.58 (m, 4H), 1.53-1.49 (m, 1H), 1.47-1.43 (m, 2H), 0.98-0.81 (m, 6H). The proton of carboxy group was not found. (ESI) m/z calcd for C₂₃H₂₉ClN₄O₃: 444.19. Found: 445.31/447.30 (M/M+2)⁺.

### Preparation of 1-(4-((5-chloropyridin-2-yl)amino)-5-(isobutyl(tetrahydro-2H-pyran-4-yl)amino)pyridin-2-yl)-N-(methylsulfonyl)cyclopropane-1-carboxamide

[chemical structure]

To a solution of 1-(4-((5-chloropyridin-2-yl)amino)-5-(isobutyl(tetrahydro-2H-pyran-4-yl)amino)pyridin-2-yl)cyclopropane-1-carboxylic acid (40 mg, 0.090 mmol) in THF, was added CDI (22 mg, 0.135 mmol), methanesulfonamide (13 mg, 0.135 mmol) and DBU (27 mg, 0.18 mmol). After the resulting mixture was stirred at room temperature for 2 hours, DCC (28 mg, 0.135 mmol) was added. After stirred at room temperature overnight, the resulting mixture was partitioned between EtOAc and H₂O. The organic layer was washed with brine, dried over Na₂SO₄, filtered and concentrated to give the crude product which was purified by HPLC (C18, 10-100% MeCN in H₂O with 0.1% formic acid) to afford the title compound (13 mg, 28% yield) as a white powder. ¹H NMR (400 MHz, DMSO) δ 8.69 (s, 1H), 8.41-8.30 (m, 3H), 7.85 (dd, J=8.8, 2.7 Hz, 1H), 7.36 (d, J=8.8 Hz, 1H), 3.88-3.81 (m, 2H), 3.25-3.20 (m, 2H), 3.11 (s, 3H), 3.00-2.86 (m, 3H), 1.84-1.75 (m, 2H), 1.57-1.44 (m, 4H), 1.43-1.30 (m, 3H), 0.85 (d, J=6.5 Hz, 6H). The proton of the sulfonamide group was not found. LCMS (ESI) m/z calcd for C₂₄H₃₂ClN₅O₄S: 521.19. Found: 522.32/524.37 (M+1)+.

### IDO1 HeLa RapidFire MS Assay

Compounds of the present invention were tested via high-throughput cellular assays utilizing detection of kynurenine via mass spectrometry and cytotoxicity as end-points. For the mass spectrometry and cytotoxicity assays, human epithelial HeLa cells (CCL-2; ATCC®, Manassas, Va.) were stimulated with human interferon-γ (IFN-γ) (Sigma-Aldrich Corporation, St. Louis, Mo.) to induce the expression of indoleamine 2, 3-dioxygenase (IDO1). Compounds with IDO1 inhibitory properties decreased the amount of kynurenine produced by the cells via the tryptophan catabolic pathway. Cellular toxicity due to the effect of compound treatment was measured using CellTiter-Glo® reagent (CTG) (Promega Corporation, Madison, Wis.), which is based on luminescent detection of ATP, an indicator of metabolically active cells.

In preparation for the assays, test compounds were serially diluted 3-fold in DMSO from a typical top concentration of 1 mM or 5 mM and plated at 0.5 μL in 384-well, polystyrene, clear bottom, tissue culture treated plates with lids (Greiner Bio-One, Kremsmünster, Austria) to generate 11-point dose response curves. Low control wells (0% kynurenine or 100% cytotoxicity) contained either 0.5 μL of DMSO in the presence of unstimulated (−IFN-γ) HeLa cells for the mass spectrometry assay or 0.5 μL of DMSO in the absence of cells for the cytotoxicity assay, and high control wells (100% kynurenine or 0% cytotoxicity) contained 0.5 μL of DMSO in the presence of stimulated (+IFN-γ) HeLa cells for both the mass spectrometry and cytotoxicity assays.

Frozen stocks of HeLa cells were washed and recovered in DMEM high glucose medium with HEPES (Thermo Fisher Scientific, Inc., Waltham, Mass.) supplemented with 10% v/v certified fetal bovine serum (FBS) (Thermo Fisher Scientific, Inc., Waltham, Mass.), and 1× penicillin-streptomycin antibiotic solution (Thermo Fisher Scientific, Inc., Waltham, Mass.). The cells were diluted to 100,000 cells/mL in the supplemented DMEM medium. 50 μL of either the cell suspension, for the mass spectrometry assay, or medium alone, for the cytotoxicity assay, were added to the low control wells, on the previously prepared 384-well compound plates, resulting in 5,000 cells/well or 0 cells/well respectively. IFN-γ was added to the remaining cell suspension at a final concentration of 10 nM, and 50 μL of the stimulated cells were added to all remaining wells on the 384-well compound plates. The plates, with lids, were then placed in a 37° C., 5% CO₂ humidified incubator for 2 days.

Following incubation, the 384-well plates were removed from the incubator and allowed to equilibrate to room temperature for 30 minutes. For the cytotoxicity assay, CellTiter-Glo® was prepared according to the manufacturer's instructions, and 10 μL were added to each plate well. After a twenty minute incubation at room temperature, luminescence was read on an EnVision® Multilabel Reader (PerkinElmer Inc., Waltham, Mass.). For the mass spectrometry assay, 10 μL of supernatant from each well of the compound-treated plates were added to 40 μL of acetonitrile, containing 10 μM of an internal standard for normalization, in 384-well, polypropylene, V-bottom plates (Greiner Bio-One, Kremsmünster, Austria) to extract the organic analytes. Following centrifugation at 2000 rpm for 10 minutes, 10 μL from each well of the acetonitrile extraction plates were added to 90 μL of sterile, distilled H₂O in 384-well, polypropylene, V-bottom plates for analysis of kynurenine and the internal standard on the RapidFire 300 (Agilent Technologies, Santa Clara, Calif.) and 4000 QTRAP MS (SCIEX, Framingham, Mass.). MS data were integrated using Agilent Technologies' RapidFire Integrator software, and data were normalized for analysis as a ratio of kynurenine to the internal standard.

The data for dose responses in the mass spectrometry assay were plotted as % IDO1 inhibition versus compound concentration following normalization using the formula 100−(100*((U−C2)/(C1-C2))), where U was the unknown value, C1 was the average of the high (100% kynurenine; 0% inhibition) control wells and C2 was the average of the low (0% kynurenine; 100% inhibition) control wells. The data for dose responses in the cytotoxicity assay were plotted as % cytotoxicity versus compound concentration following normalization using the formula 100−(100*((U−C2)/(C1-C2))), where U was the unknown value, C1 was the average of the high (0% cytotoxicity) control wells and C2 was the average of the low (100% cytotoxicity) control wells.

Curve fitting was performed with the equation y=A+((B−A)/(1+(10×/10C)D)), where

A was the minimum response, B was the maximum response, C was the log(XC50) and D was the Hill slope. The results for each test compound were recorded as pIC50 values for the mass spectrometry assay and as pCC50 values for the cytoxicity assay (−C in the above equation).

### IDO1 PBMC RapidFire MS Assay

Compounds of the present invention were tested via high-throughput cellular assays utilizing detection of kynurenine via mass spectrometry and cytotoxicity as end-points. For the mass spectrometry and cytotoxicity assays, human peripheral blood mononuclear cells (PBMC) (PB003F; AllCells®, Alameda, Calif.) were stimulated with human interferon-γ (IFN-γ) (Sigma-Aldrich Corporation, St. Louis, Mo.) and lipopolysaccharide from Salmonella minnesota (LPS) (Invivogen, San Diego, Calif.) to induce the expression of indoleamine 2, 3-dioxygenase (IDO1). Compounds with IDO1 inhibitory properties decreased the amount of kynurenine produced by the cells via the tryptophan catabolic pathway. Cellular toxicity due to the effect of compound treatment was measured using CellTiter-Glo® reagent (CTG) (Promega Corporation, Madison, Wis.), which is based on luminescent detection of ATP, an indicator of metabolically active cells.

In preparation for the assays, test compounds were serially diluted 3-fold in DMSO from a typical top concentration of 1 mM or 5 mM and plated at 0.5 μL in 384-well, polystyrene, clear bottom, tissue culture treated plates with lids (Greiner Bio-One, Kremsmünster, Austria) to generate 11-point dose response curves. Low control wells (0% kynurenine or 100% cytotoxicity) contained either 0.5 μL of DMSO in the presence of unstimulated (−IFN-γ/-LPS) PBMCs for the mass spectrometry assay or 0.5 μL of DMSO in the absence of cells for the cytotoxicity assay, and high control wells (100% kynurenine or 0% cytotoxicity) contained 0.5 μL of DMSO in the presence of stimulated (+IFN-γ/+LPS) PBMCs for both the mass spectrometry and cytotoxicity assays.

Frozen stocks of PBMCs were washed and recovered in RPMI 1640 medium (Thermo Fisher Scientific, Inc., Waltham, Mass.) supplemented with 10% v/v heat-inactivated fetal bovine serum (FBS) (Thermo Fisher Scientific, Inc., Waltham, Mass.), and 1× penicillin-streptomycin antibiotic solution (Thermo Fisher Scientific, Inc., Waltham, Mass.). The cells were diluted to 1,000,000 cells/mL in the supplemented RPMI 1640 medium. 50 μL of either the cell suspension, for the mass spectrometry assay, or medium alone, for the cytotoxicity assay, were added to the low control wells, on the previously prepared 384-well compound plates, resulting in 50,000 cells/well or 0 cells/well respectively. IFN-γ and LPS were added to the remaining cell suspension at final concentrations of 100 ng/ml and 50 ng/ml respectively, and 50 μL of the stimulated cells were added to all remaining wells on the 384-well compound plates. The plates, with lids, were then placed in a 37° C., 5% CO₂ humidified incubator for 2 days.

Following incubation, the 384-well plates were removed from the incubator and allowed to equilibrate to room temperature for 30 minutes. For the cytotoxicity assay, CellTiter-Glo® was prepared according to the manufacturer's instructions, and 40 μL were added to each plate well. After a twenty minute incubation at room temperature, luminescence was read on an EnVision® Multilabel Reader (PerkinElmer Inc., Waltham, Mass.). For the mass spectrometry assay, 10 μL of supernatant from each well of the compound-treated plates were added to 40 μL of acetonitrile, containing 10 μM of an internal standard for normalization, in 384-well, polypropylene, V-bottom plates (Greiner Bio-One, Kremsmünster, Austria) to extract the organic analytes. Following centrifugation at 2000 rpm for 10 minutes, 10 μL from each well of the acetonitrile extraction plates were added to 90 μL of sterile, distilled H₂O in 384-well, polypropylene, V-bottom plates for analysis of kynurenine and the internal standard on the RapidFire 300 (Agilent Technologies, Santa Clara, Calif.) and 4000 QTRAP MS (SCIEX, Framingham, Mass.). MS data were integrated using Agilent Technologies' RapidFire Integrator software, and data were normalized for analysis as a ratio of kynurenine to the internal standard.

The data for dose responses in the mass spectrometry assay were plotted as % IDO1 inhibition versus compound concentration following normalization using the formula 100-(100*((U−C2)/(C1-C2))), where U was the unknown value, C1 was the average of the high (100% kynurenine; 0% inhibition) control wells and C2 was the average of the low (0% kynurenine; 100% inhibition) control wells. The data for dose responses in the cytotoxicity assay were plotted as % cytotoxicity versus compound concentration following normalization using the formula 100-(100*((U−C2)/(C1-C2))), where U was the unknown value, C1 was the average of the high (0% cytotoxicity) control wells and C2 was the average of the low (100% cytotoxicity) control wells.

Curve fitting was performed with the equation y=A+((B−A)/(1+(10×/10C)D)), where A was the minimum response, B was the maximum response, C was the log(XC50) and D was the Hill slope. The results for each test compound were recorded as pIC50 values for the mass spectrometry assay and as pCC50 values for the cytoxicity assay (−C in the above equation).

TABLE 1
IDO1 potency of compounds in PBMC or HeLa assay

| patent | IDO1 PBMC | IDO1 HeLa |
|---|---|---|
| example | pIC₅₀ | pIC₅₀ |
| 1 | 8.5 | |
| 2 | 8.8 | |
| 3 | 7.7 | |
| 4 | 9.1 | |
| 5 | 8.2 | |
| 6 | 8.2 | |
| 7 | 8.3 | |
| 8 | 7.7 | |
| 9 | 8.1 | |
| 10 | 8.3 | |
| 11 | 7.5 | |
| 12 | 7.6 | |
| 13 | 7.9 | |
| 14 | 7.8 | |
| 15 | n/a | 6.8 |
| 16 | n/a | 8.0 |
| 17 | 8.2 | |
| 18 | 8.5 | |
| 19 | 8.3 | |
| 20 | 7.9 | |
| 21 | 7.4 | |
| 22 | <5 | |
| 23 | 7.3 | |
| 24 | 8.6 | |
| 25 | 7.3 | |

1. A compound of Formula I [chemical structure]
Formula I
or a pharmaceutically acceptable salt thereof, wherein:
each X is CH or one X is N and the other two are CH;
R¹ and R² are independently H or C1-3alkyl, or R¹ and R² may join together with the carbon atom to which they are bonded to form a 3-6 membered cycloalkyl;
R³ is CO₂H or an acid isostere;
R⁴ is a 5 or 6-membered heterocycle or heteroaryl containing 1 to 4 heteroatoms selected from N, S, and O, wherein said heterocycle or heteroaryl may optionally be substituted by 1 or 2 substituent selected from the group consisting of halogen, C₃₋₆cycloalkyl, CH₂OH, C(O)NH₂, CN, CH₂OC₁₋₃alkyl, C₁₋₃alkyl optionally substituted by 1-3 halogens, and wherein said CH₂OH is optionally converted into a prodrug by converting the CH₂OH group to a CH₂OC(O)CH₃, CH₂OC(O)C(C₁₋₄alkyl)₃, or OP(O)(OH)₂ group, or OP(O)(OC₁₋₄alkyl)₂ group; and
R⁵ is a 4, 5, or 6-membered cycloalkyl optionally substituted with an OH or a OCH₃ group or 1 or 2 halogens, or a 5 or 6-membered heterocycle containing an O or a N optionally substituted with a substituent selected from the group consisting of halogen, OH, C₁₋₄alkyl; OC₁₋₃alkyl, C(O)C₃₋₆cycloalkyl, BOC, C(O)C₁₋₃alkyl-O—C₁₋₃alkyl; C(O)C₁₋₃alkyl; C(O)—O—C₁₋₃alkyl, and a 4 to 6-membered heterocycle or heteroaryl containing 1 to 4 heteroatoms selected from N, S, and O, wherein said heterocycle or heteroaryl may optionally be substituted by 1 substituent selected from the group consisting of halogen, C₃₋₆cycloalkyl, CH₂OH, C(O)NH₂, CN, CH₂OC₁₋₃alkyl, C₁₋₃alkyl optionally substituted by 1-3 halogens.

2. A compound or salt according to claim 1 wherein R¹ and R² are independently H or CH₃, or R¹ and R² together with the carbon to which they are bonded form a cyclopropyl ring.

3. A compound or salt according to claim 1 wherein R³ is CO₂H, —C(O)—NH—S(O)₂—CF₃, or —C(O)—NH—S(O)₂—CH₃.

4. A compound or salt according to claim 1 wherein R⁴ is a pyridine, thiadiazole, pyrimidine, pyrazine, pyridazine, triazol, or thiazol, optionally substituted with 1 or 2 substituent selected from the group consisting of F, Cl, and cyclopropyl.

5. A compound or salt according to claim 1 wherein R⁵ is C₁₋₄alkyl or a 6-membered heterocycle containing an O or a N.

6. A compound or salt according to claim 5 wherein R⁵ is unsubstituted.

7. A compound or salt according to claim 1 wherein R¹ and R² are independently H or CH₃, or R¹ and R² together with the carbon to which they are bonded form a cyclopropyl ring; R³ is CO₂H, —C(O)—NH—S(O)₂—CF₃, or —C(O)—NH—S(O)₂—CH₃; R⁴ is a pyridine, thiadiazole, pyrimidine, pyrazine, pyridazine, triazol, or thiazol, optionally substituted with 1 or 2 substituent selected from the group consisting of F, Cl, and cyclopropyl; and R⁵ is C₁₋₄alkyl or a 6-membered heterocycle containing an O or a N.

8. A pharmaceutical composition comprising a compound or salt according to claim 1.

9. A method of treating a disease or condition that would benefit from inhibition of IDO1 comprising the step of administration of a composition according to claim 8.

10. The method of claim 9 wherein in said disease or condition, biomarkers of IDO activity are elevated.

11. The method of claim 9 wherein said biomarkers are plasma kynurenine or the plasma kynurenine/tryptophan ratio.

12. The method of claim 9 wherein said disease or condition is chronic viral infection; chronic bacterial infections; cancer; sepsis; or a neurological disorder.

13. The method of claim 9 wherein said chronic viral infections are those involving HIV, HBV, or HCV; said chronic bacterial infections are tuberculosis or prosthetic joint infection; and said neurological disorders are major depressive disorder, Huntington's disease, or Parkinson's disease.

14. The method of claim 13 wherein said disease or condition is inflammation associated with HIV infection; chronic viral infections involving hepatitis B virus or hepatitis C virus; cancer; or sepsis.

15-16. (canceled)
